SEQUENCE LISTING (377 sequences)

SEQ ID NO: 1 - protein, 12 aa, Severe acute respiratory syndrome coronavirus 2
TEPKKDKKKKAD

SEQ ID NO: 2 - protein, 12 aa, Severe acute respiratory syndrome coronavirus 2
VRDPQTLEILDI

SEQ ID NO: 3 - protein, 12 aa, Severe acute respiratory syndrome coronavirus 2
FPPTEPKKDKKK

SEQ ID NO: 4 - protein, 12 aa, Severe acute respiratory syndrome coronavirus 2
TDAVRDPQTLEI

SEQ ID NO: 5 - protein, 12 aa, Severe acute respiratory syndrome coronavirus 2
AAEASKKPRQKR

SEQ ID NO: 6 - protein, 12 aa, Severe acute respiratory syndrome coronavirus 2
CCKFDEDDSEPV

SEQ ID NO: 7 - protein, 12 aa, Severe acute respiratory syndrome coronavirus 2
PFQQFGRDIADT

SEQ ID NO: 8 - protein, 12 aa, Severe acute respiratory syndrome coronavirus 2
DDDSQQTVGQQD

SEQ ID NO: 9 - protein, 12 aa, Severe acute respiratory syndrome coronavirus 2
LQPEEEQEEDWL

SEQ ID NO: 10 - protein, 12 aa, Severe acute respiratory syndrome coronavirus 2
DDDYFNKKDWYD

SEQ ID NO: 11 - protein, 12 aa, Severe acute respiratory syndrome coronavirus 2
QPELDSFKEELD

SEQ ID NO: 12 - protein, 12 aa, Severe acute respiratory syndrome coronavirus 2
AEASKKPRQKRT

SEQ ID NO: 13 - protein, 12 aa, Severe acute respiratory syndrome coronavirus 2
FDEDDSEPVLKG

SEQ ID NO: 14 - protein, 12 aa, Severe acute respiratory syndrome coronavirus 2
TEDDYQGKPLEF

SEQ ID NO: 15 - protein, 12 aa, Severe acute respiratory syndrome coronavirus 2
YNENGTITDAVD

SEQ ID NO: 16 - protein, 12 aa, Severe acute respiratory syndrome coronavirus 2
EGDCEEEEFEPS

SEQ ID NO: 17 - protein, 12 aa, Severe acute respiratory syndrome coronavirus 2
LGDELGTDPYED

SEQ ID NO: 18 - protein, 12 aa, Severe acute respiratory syndrome coronavirus 2
QFGRDIADTTDA

SEQ ID NO: 19 - protein, 12 aa, Severe acute respiratory syndrome coronavirus 2
QQQQGQTVTKKS

SEQ ID NO: 20 - protein, 12 aa, Severe acute respiratory syndrome coronavirus 2
PDEDEEEGDCEE

SEQ ID NO: 21 - protein, 12 aa, Severe acute respiratory syndrome coronavirus 2
ETTADIVVFDEI

SEQ ID NO: 22 - protein, 12 aa, Severe acute respiratory syndrome coronavirus 2
ADTTDAVRDPQT

SEQ ID NO: 23 - protein, 12 aa, Severe acute respiratory syndrome coronavirus 2
QLPPAYTNSFTR

SEQ ID NO: 24 - protein, 12 aa, Severe acute respiratory syndrome coronavirus 2
PAYTNSFTRGVY

SEQ ID NO: 25 - protein, 12 aa, Severe acute respiratory syndrome coronavirus 2
TNSFTRGVYYPD

SEQ ID NO: 26 - protein, 12 aa, Severe acute respiratory syndrome coronavirus 2
FTRGVYYPDKVF

SEQ ID NO: 27 - protein, 12 aa, Severe acute respiratory syndrome coronavirus 2
GVYYPDKVFRSS

SEQ ID NO: 28 - protein, 12 aa, Severe acute respiratory syndrome coronavirus 2
YPDKVFRSSVLH

SEQ ID NO: 29 - protein, 12 aa, Severe acute respiratory syndrome coronavirus 2
RTFLLKYNENGT

SEQ ID NO: 30 - protein, 12 aa, Severe acute respiratory syndrome coronavirus 2
LLKYNENGTITD

SEQ ID NO: 31 - protein, 12 aa, Severe acute respiratory syndrome coronavirus 2
NGTITDAVDCAL

SEQ ID NO: 32 - protein, 12 aa, Severe acute respiratory syndrome coronavirus 2
ITDAVDCALDPL

SEQ ID NO: 33 - protein, 12 aa, Severe acute respiratory syndrome coronavirus 2
AVDCALDPLSET

SEQ ID NO: 34 - protein, 12 aa, Severe acute respiratory syndrome coronavirus 2
DCALDPLSETKC

SEQ ID NO: 35 - protein, 12 aa, Severe acute respiratory syndrome coronavirus 2
CALDPLSETKCT

SEQ ID NO: 36 - protein, 12 aa, Severe acute respiratory syndrome coronavirus 2
SETKCTLKSFTV

SEQ ID NO: 37 - protein, 12 aa, Severe acute respiratory syndrome coronavirus 2
KCTLKSFTVEKG

SEQ ID NO: 38 - protein, 12 aa, Severe acute respiratory syndrome coronavirus 2
RFPNITNLCPFG

SEQ ID NO: 39 - protein, 12 aa, Severe acute respiratory syndrome coronavirus 2
NLCPFGEVFNAT

SEQ ID NO: 40 - protein, 12 aa, Severe acute respiratory syndrome coronavirus 2
EVFNATRFASVY

SEQ ID NO: 41 - protein, 12 aa, Severe acute respiratory syndrome coronavirus 2
RFASVYAWNRKR

SEQ ID NO: 42 - protein, 12 aa, Severe acute respiratory syndrome coronavirus 2
AWNRKRISNCVA

SEQ ID NO: 43 - protein, 12 aa, Severe acute respiratory syndrome coronavirus 2
ISNCVADYSVLY

SEQ ID NO: 44 - protein, 12 aa, Severe acute respiratory syndrome coronavirus 2
DYSVLYNSASFS

SEQ ID NO: 45 - protein, 12 aa, Severe acute respiratory syndrome coronavirus 2
NSASFSTFKCYG

SEQ ID NO: 46 - protein, 12 aa, Severe acute respiratory syndrome coronavirus 2
TFKCYGVSPTKL

SEQ ID NO: 47 - protein, 12 aa, Severe acute respiratory syndrome coronavirus 2
VSPTKLNDLCFT

SEQ ID NO: 48 - protein, 12 aa, Severe acute respiratory syndrome coronavirus 2
NDLCFTNVYADS

SEQ ID NO: 49 - protein, 12 aa, Severe acute respiratory syndrome coronavirus 2
NVYADSFVIRGD

SEQ ID NO: 50 - protein, 12 aa, Severe acute respiratory syndrome coronavirus 2
FVIRGDEVRQIA

SEQ ID NO: 51 - protein, 12 aa, Severe acute respiratory syndrome coronavirus 2
EVRQIAPGQTGK

SEQ ID NO: 52 - protein, 12 aa, Severe acute respiratory syndrome coronavirus 2
PGQTGKIADYNY

SEQ ID NO: 53 - protein, 12 aa, Severe acute respiratory syndrome coronavirus 2
IADYNYKLPDDF

SEQ ID NO: 54 - protein, 12 aa, Severe acute respiratory syndrome coronavirus 2
KLPDDFTGCVIA

SEQ ID NO: 55 - protein, 12 aa, Severe acute respiratory syndrome coronavirus 2
TGCVIAWNSNNL

SEQ ID NO: 56 - protein, 12 aa, Severe acute respiratory syndrome coronavirus 2
WNSNNLDSKVGG

SEQ ID NO: 57 - protein, 12 aa, Severe acute respiratory syndrome coronavirus 2
DSKVGGNYNYLY

SEQ ID NO: 58 - protein, 12 aa, Severe acute respiratory syndrome coronavirus 2
NYNYLYRLFRKS

SEQ ID NO: 59 - protein, 12 aa, Severe acute respiratory syndrome coronavirus 2
RLFRKSNLKPFE

SEQ ID NO: 60 - protein, 12 aa, Severe acute respiratory syndrome coronavirus 2
NLKPFERDISTE

SEQ ID NO: 61 - protein, 12 aa, Severe acute respiratory syndrome coronavirus 2
RDISTEIYQAGS

SEQ ID NO: 62 - protein, 12 aa, Severe acute respiratory syndrome coronavirus 2
IYQAGSTPCNGV

SEQ ID NO: 63 - protein, 12 aa, Severe acute respiratory syndrome coronavirus 2
TPCNGVEGFNCY

SEQ ID NO: 64 - protein, 12 aa, Severe acute respiratory syndrome coronavirus 2
EGFNCYFPLQSY

SEQ ID NO: 65 - protein, 12 aa, Severe acute respiratory syndrome coronavirus 2
FPLQSYGFQPTN

SEQ ID NO: 66 - protein, 12 aa, Severe acute respiratory syndrome coronavirus 2
GFQPTNGVGYQP

SEQ ID NO: 67 - protein, 12 aa, Severe acute respiratory syndrome coronavirus 2
GVGYQPYRVVVL

SEQ ID NO: 68 - protein, 12 aa, Severe acute respiratory syndrome coronavirus 2
YRVVVLSFELLH

SEQ ID NO: 69 - protein, 12 aa, Severe acute respiratory syndrome coronavirus 2
SFELLHAPATVC

SEQ ID NO: 70 - protein, 12 aa, Severe acute respiratory syndrome coronavirus 2
APATVCGPKKST

SEQ ID NO: 71 - protein, 12 aa, Severe acute respiratory syndrome coronavirus 2
TVCGPKKSTNLV

SEQ ID NO: 72 - protein, 12 aa, Severe acute respiratory syndrome coronavirus 2
LTGTGVLTESNK

SEQ ID NO: 73 - protein, 12 aa, Severe acute respiratory syndrome coronavirus 2
TGVLTESNKKFL

SEQ ID NO: 74 - protein, 12 aa, Severe acute respiratory syndrome coronavirus 2
LTESNKKFLPFQ

SEQ ID NO: 75 - protein, 12 aa, Severe acute respiratory syndrome coronavirus 2
SNKKFLPFQQFG

SEQ ID NO: 76 - protein, 12 aa, Severe acute respiratory syndrome coronavirus 2
KFLPFQQFGRDI

SEQ ID NO: 77 - protein, 12 aa, Severe acute respiratory syndrome coronavirus 2
RDIADTTDAVRD

SEQ ID NO: 78 - protein, 12 aa, Severe acute respiratory syndrome coronavirus 2
TTDAVRDPQTLE

SEQ ID NO: 79 - protein, 12 aa, Severe acute respiratory syndrome coronavirus 2
DPQTLEILDITP

SEQ ID NO: 80 - protein, 12 aa, Severe acute respiratory syndrome coronavirus 2
PQTLEILDITPC

SEQ ID NO: 81 - protein, 12 aa, Severe acute respiratory syndrome coronavirus 2
LEILDITPCSFG

SEQ ID NO: 82 - protein, 12 aa, Severe acute respiratory syndrome coronavirus 2
ILDITPCSFGGV

SEQ ID NO: 83 - protein, 12 aa, Severe acute respiratory syndrome coronavirus 2
LDITPCSFGGVS

SEQ ID NO: 84 - protein, 12 aa, Severe acute respiratory syndrome coronavirus 2
TPCSFGGVSVIT

SEQ ID NO: 85 - protein, 12 aa, Severe acute respiratory syndrome coronavirus 2
SFGGVSVITPGT

SEQ ID NO: 86 - protein, 12 aa, Severe acute respiratory syndrome coronavirus 2
GVSVITPGTNTS

SEQ ID NO: 87 - protein, 12 aa, Severe acute respiratory syndrome coronavirus 2
VITPGTNTSNQV

SEQ ID NO: 88 - protein, 12 aa, Severe acute respiratory syndrome coronavirus 2
PGTNTSNQVAVL

SEQ ID NO: 89 - protein, 12 aa, Severe acute respiratory syndrome coronavirus 2
NTSNQVAVLYQD

SEQ ID NO: 90 - protein, 12 aa, Severe acute respiratory syndrome coronavirus 2
NQVAVLYQDVNC

SEQ ID NO: 91 - protein, 12 aa, Severe acute respiratory syndrome coronavirus 2
AVLYQDVNCTEV

SEQ ID NO: 92 - protein, 12 aa, Severe acute respiratory syndrome coronavirus 2
VLYQDVNCTEVP

SEQ ID NO: 93 - protein, 12 aa, Severe acute respiratory syndrome coronavirus 2
KRSFIEDLLFNK

SEQ ID NO: 94 - protein, 12 aa, Severe acute respiratory syndrome coronavirus 2
FIEDLLFNKVTL

SEQ ID NO: 95 - protein, 12 aa, Severe acute respiratory syndrome coronavirus 2
DLLFNKVTLADA

SEQ ID NO: 96 - protein, 12 aa, Severe acute respiratory syndrome coronavirus 2
FNKVTLADAGFI

SEQ ID NO: 97 - protein, 12 aa, Severe acute respiratory syndrome coronavirus 2
VTLADAGFIKQY

SEQ ID NO: 98 - protein, 12 aa, Severe acute respiratory syndrome coronavirus 2
ADAGFIKQYGDC

SEQ ID NO: 99 - protein, 12 aa, Severe acute respiratory syndrome coronavirus 2
DAGFIKQYGDCL

SEQ ID NO: 100 - protein, 12 aa, Severe acute respiratory syndrome coronavirus 2
ALGKLQDVVNQN

SEQ ID NO: 101 - protein, 12 aa, Severe acute respiratory syndrome coronavirus 2
KLQDVVNQNAQA

SEQ ID NO: 102 - protein, 12 aa, Severe acute respiratory syndrome coronavirus 2
DVVNQNAQALNT

SEQ ID NO: 103 - protein, 12 aa, Severe acute respiratory syndrome coronavirus 2
NQNAQALNTLVK

SEQ ID NO: 104 - protein, 12 aa, Severe acute respiratory syndrome coronavirus 2
AQALNTLVKQLS

SEQ ID NO: 105 - protein, 12 aa, Severe acute respiratory syndrome coronavirus 2
LNTLVKQLSSNF

SEQ ID NO: 106 - protein, 12 aa, Severe acute respiratory syndrome coronavirus 2
TLVKQLSSNFGA

SEQ ID NO: 107 - protein, 12 aa, Severe acute respiratory syndrome coronavirus 2
DPLQPELDSFKE

SEQ ID NO: 108 - protein, 12 aa, Severe acute respiratory syndrome coronavirus 2
LDSFKEELDKYF

SEQ ID NO: 109 - protein, 12 aa, Severe acute respiratory syndrome coronavirus 2
FKEELDKYFKNH

SEQ ID NO: 110 - protein, 12 aa, Severe acute respiratory syndrome coronavirus 2
ELDKYFKNHTSP

SEQ ID NO: 111 - protein, 12 aa, Severe acute respiratory syndrome coronavirus 2
DKYFKNHTSPDV

SEQ ID NO: 112 - protein, 12 aa, Severe acute respiratory syndrome coronavirus 2
GINASVVNIQKE

SEQ ID NO: 113 - protein, 12 aa, Severe acute respiratory syndrome coronavirus 2
ASVVNIQKEIDR

SEQ ID NO: 114 - protein, 12 aa, Severe acute respiratory syndrome coronavirus 2
VNIQKEIDRLNE

SEQ ID NO: 115 - protein, 12 aa, Severe acute respiratory syndrome coronavirus 2
QKEIDRLNEVAK

SEQ ID NO: 116 - protein, 12 aa, Severe acute respiratory syndrome coronavirus 2
IDRLNEVAKNLN

SEQ ID NO: 117 - protein, 12 aa, Severe acute respiratory syndrome coronavirus 2
LNEVAKNLNESL

SEQ ID NO: 118 - protein, 12 aa, Severe acute respiratory syndrome coronavirus 2
EVAKNLNESLID

SEQ ID NO: 119 - protein, 12 aa, Severe acute respiratory syndrome coronavirus 2
CCSCGSCCKFDE

SEQ ID NO: 120 - protein, 12 aa, Severe acute respiratory syndrome coronavirus 2
CGSCCKFDEDDS

SEQ ID NO: 121 - protein, 12 aa, Severe acute respiratory syndrome coronavirus 2
DDSEPVLKGVKL

SEQ ID NO: 122 - protein, 12 aa, Severe acute respiratory syndrome coronavirus 2
EPVLKGVKLHYT

SEQ ID NO: 123 - protein, 12 aa, Severe acute respiratory syndrome coronavirus 2
PRITFGGPSDST

SEQ ID NO: 124 - protein, 12 aa, Severe acute respiratory syndrome coronavirus 2
TFGGPSDSTGSN

SEQ ID NO: 125 - protein, 12 aa, Severe acute respiratory syndrome coronavirus 2
GPSDSTGSNQNG

SEQ ID NO: 126 - protein, 12 aa, Severe acute respiratory syndrome coronavirus 2
DSTGSNQNGERS

SEQ ID NO: 127 - protein, 12 aa, Severe acute respiratory syndrome coronavirus 2
GSNQNGERSGAR

SEQ ID NO: 128 - protein, 12 aa, Severe acute respiratory syndrome coronavirus 2
QNGERSGARSKQ

SEQ ID NO: 129 - protein, 12 aa, Severe acute respiratory syndrome coronavirus 2
ERSGARSKQRRP

SEQ ID NO: 130 - protein, 12 aa, Severe acute respiratory syndrome coronavirus 2
GARSKQRRPQGL

SEQ ID NO: 131 - protein, 12 aa, Severe acute respiratory syndrome coronavirus 2
RSKQRRPQGLPN

SEQ ID NO: 132 - protein, 12 aa, Severe acute respiratory syndrome coronavirus 2
SQASSRSSSRSR

SEQ ID NO: 133 - protein, 12 aa, Severe acute respiratory syndrome coronavirus 2
SSRSSSRSRNSS

SEQ ID NO: 134 - protein, 12 aa, Severe acute respiratory syndrome coronavirus 2
SSSRSRNSSRNS

SEQ ID NO: 135 - protein, 12 aa, Severe acute respiratory syndrome coronavirus 2
RSRNSSRNSTPG

SEQ ID NO: 136 - protein, 12 aa, Severe acute respiratory syndrome coronavirus 2
NSSRNSTPGSSR

SEQ ID NO: 137 - protein, 12 aa, Severe acute respiratory syndrome coronavirus 2
RNSTPGSSRGTS

SEQ ID NO: 138 - protein, 12 aa, Severe acute respiratory syndrome coronavirus 2
NSTPGSSRGTSP

SEQ ID NO: 139 - protein, 12 aa, Severe acute respiratory syndrome coronavirus 2
KMSGKGQQQQGQ

SEQ ID NO: 140 - protein, 12 aa, Severe acute respiratory syndrome coronavirus 2
TVTKKSAAEASK

SEQ ID NO: 141 - protein, 12 aa, Severe acute respiratory syndrome coronavirus 2
YKTFPPTEPKKD

SEQ ID NO: 142 - protein, 12 aa, Severe acute respiratory syndrome coronavirus 2
KKDKKKKADETQ

SEQ ID NO: 143 - protein, 12 aa, Severe acute respiratory syndrome coronavirus 2
KKKKADETQALP

SEQ ID NO: 144 - protein, 12 aa, Severe acute respiratory syndrome coronavirus 2
KADETQALPQRQ

SEQ ID NO: 145 - protein, 12 aa, Severe acute respiratory syndrome coronavirus 2
ADETQALPQRQK

SEQ ID NO: 146 - protein, 12 aa, Severe acute respiratory syndrome coronavirus 2
ETQALPQRQKKQ

SEQ ID NO: 147 - protein, 12 aa, Severe acute respiratory syndrome coronavirus 2
QALPQRQKKQQT

SEQ ID NO: 148 - protein, 12 aa, Severe acute respiratory syndrome coronavirus 2
GRCDIKDLPKEI

SEQ ID NO: 149 - protein, 12 aa, Severe acute respiratory syndrome coronavirus 2
DLPKEITVATSR

SEQ ID NO: 150 - protein, 12 aa, Severe acute respiratory syndrome coronavirus 2
TVATSRTLSYYK

SEQ ID NO: 151 - protein, 12 aa, Severe acute respiratory syndrome coronavirus 2
TSRTLSYYKLGA

SEQ ID NO: 152 - protein, 12 aa, Severe acute respiratory syndrome coronavirus 2
TDPYEDFQENWN

SEQ ID NO: 153 - protein, 12 aa, Severe acute respiratory syndrome coronavirus 2
FQENWNTKHSSG

SEQ ID NO: 154 - protein, 12 aa, Severe acute respiratory syndrome coronavirus 2
TKHSSGVTRELM

SEQ ID NO: 155 - protein, 12 aa, Severe acute respiratory syndrome coronavirus 2
KHSSGVTRELMR

SEQ ID NO: 156 - protein, 12 aa, Severe acute respiratory syndrome coronavirus 2
KRGVYCCREHEH

SEQ ID NO: 157 - protein, 12 aa, Severe acute respiratory syndrome coronavirus 2
CREHEHEIAWYT

SEQ ID NO: 158 - protein, 12 aa, Severe acute respiratory syndrome coronavirus 2
EIAWYTERSEKS

SEQ ID NO: 159 - protein, 12 aa, Severe acute respiratory syndrome coronavirus 2
ERSEKSYELQTP

SEQ ID NO: 160 - protein, 12 aa, Severe acute respiratory syndrome coronavirus 2
RSEKSYELQTPF

SEQ ID NO: 161 - protein, 12 aa, Severe acute respiratory syndrome coronavirus 2
ASHMYCSFYPPD

SEQ ID NO: 162 - protein, 12 aa, Severe acute respiratory syndrome coronavirus 2
YCSFYPPDEDEE

SEQ ID NO: 163 - protein, 12 aa, Severe acute respiratory syndrome coronavirus 2
SFYPPDEDEEEG

SEQ ID NO: 164 - protein, 12 aa, Severe acute respiratory syndrome coronavirus 2
EDEEEGDCEEEE

SEQ ID NO: 165 - protein, 12 aa, Severe acute respiratory syndrome coronavirus 2
DCEEEEFEPSTQ

SEQ ID NO: 166 - protein, 12 aa, Severe acute respiratory syndrome coronavirus 2
EEFEPSTQYEYG

SEQ ID NO: 167 - protein, 12 aa, Severe acute respiratory syndrome coronavirus 2
FEPSTQYEYGTE

SEQ ID NO: 168 - protein, 12 aa, Severe acute respiratory syndrome coronavirus 2
TQYEYGTEDDYQ

SEQ ID NO: 169 - protein, 12 aa, Severe acute respiratory syndrome coronavirus 2
YEYGTEDDYQGK

SEQ ID NO: 170 - protein, 12 aa, Severe acute respiratory syndrome coronavirus 2
EYGTEDDYQGKP

SEQ ID NO: 171 - protein, 12 aa, Severe acute respiratory syndrome coronavirus 2
GKPLEFGATSAA

SEQ ID NO: 172 - protein, 12 aa, Severe acute respiratory syndrome coronavirus 2
EFGATSAALQPE

SEQ ID NO: 173 - protein, 12 aa, Severe acute respiratory syndrome coronavirus 2
GATSAALQPEEE

SEQ ID NO: 174 - protein, 12 aa, Severe acute respiratory syndrome coronavirus 2
AALQPEEEQEED

SEQ ID NO: 175 - protein, 12 aa, Severe acute respiratory syndrome coronavirus 2
EEQEEDWLDDDS

SEQ ID NO: 176 - protein, 12 aa, Severe acute respiratory syndrome coronavirus 2
QEEDWLDDDSQQ

SEQ ID NO: 177 - protein, 12 aa, Severe acute respiratory syndrome coronavirus 2
WLDDDSQQTVGQ

SEQ ID NO: 178 - protein, 12 aa, Severe acute respiratory syndrome coronavirus 2
SQQTVGQQDGSE

SEQ ID NO: 179 - protein, 12 aa, Severe acute respiratory syndrome coronavirus 2
GPITDVFYKENS

SEQ ID NO: 180 - protein, 12 aa, Severe acute respiratory syndrome coronavirus 2
FYKENSYTTTIK

SEQ ID NO: 181 - protein, 12 aa, Severe acute respiratory syndrome coronavirus 2
YTTTIKPVTYKL

SEQ ID NO: 182 - protein, 12 aa, Severe acute respiratory syndrome coronavirus 2
PVTYKLDGVVCT

SEQ ID NO: 183 - protein, 12 aa, Severe acute respiratory syndrome coronavirus 2
VTYKLDGVVCTE

SEQ ID NO: 184 - protein, 12 aa, Severe acute respiratory syndrome coronavirus 2
KKDWYDFVENPD

SEQ ID NO: 185 - protein, 12 aa, Severe acute respiratory syndrome coronavirus 2
FVENPDILRVYA

SEQ ID NO: 186 - protein, 12 aa, Severe acute respiratory syndrome coronavirus 2
ILRVYANLGERV

SEQ ID NO: 187 - protein, 12 aa, Severe acute respiratory syndrome coronavirus 2
LRVYANLGERVR

SEQ ID NO: 188 - protein, 12 aa, Severe acute respiratory syndrome coronavirus 2
MLTNDNTSRYWE

SEQ ID NO: 189 - protein, 12 aa, Severe acute respiratory syndrome coronavirus 2
TSRYWEPEFYEA

SEQ ID NO: 190 - protein, 12 aa, Severe acute respiratory syndrome coronavirus 2
PEFYEAMYTPHT

SEQ ID NO: 191 - protein, 12 aa, Severe acute respiratory syndrome coronavirus 2
MYTPHTVLQAVG

SEQ ID NO: 192 - protein, 12 aa, Severe acute respiratory syndrome coronavirus 2
DIVVFDEISMAT

SEQ ID NO: 193 - protein, 12 aa, Severe acute respiratory syndrome coronavirus 2
CRRCPAEIVDTV

SEQ ID NO: 194 - protein, 12 aa, Severe acute respiratory syndrome coronavirus 2
EIVDTVSALVYD

SEQ ID NO: 195 - protein, 12 aa, Severe acute respiratory syndrome coronavirus 2
SALVYDNKLKAH

SEQ ID NO: 196 - protein, 12 aa, Severe acute respiratory syndrome coronavirus 2
NKLKAHKDKSAQ

SEQ ID NO: 197 - protein, 12 aa, Severe acute respiratory syndrome coronavirus 2
PTQTVDSSQGSE

SEQ ID NO: 198 - protein, 12 aa, Severe acute respiratory syndrome coronavirus 2
SSQGSEYDYVIF

SEQ ID NO: 199 - protein, 12 aa, Severe acute respiratory syndrome coronavirus 2
YDYVIFTQTTET

SEQ ID NO: 200 - protein, 12 aa, Severe acute respiratory syndrome coronavirus 2
TQTTETAHSCNV

SEQ ID NO: 201 - protein, 12 aa, Severe acute respiratory syndrome coronavirus 2
TTETAHSCNVNR

SEQ ID NO: 202 - protein, 12 aa, Severe acute respiratory syndrome coronavirus 2
GLAKRFKESPFE

SEQ ID NO: 203 - protein, 12 aa, Severe acute respiratory syndrome coronavirus 2
KESPFELEDFIP

SEQ ID NO: 204 - protein, 12 aa, Severe acute respiratory syndrome coronavirus 2
LEDFIPMDSTVK

SEQ ID NO: 205 - protein, 12 aa, Severe acute respiratory syndrome coronavirus 2
MDSTVKNYFITD

SEQ ID NO: 206 - protein, 12 aa, Severe acute respiratory syndrome coronavirus 2
DSTVKNYFITDA

SEQ ID NO: 207 - protein, 12 aa, Severe acute respiratory syndrome coronavirus 2
SGVVNPVMEPIY

SEQ ID NO: 208 - protein, 12 aa, Severe acute respiratory syndrome coronavirus 2
VMEPIYDEPTTT

SEQ ID NO: 209 - protein, 12 aa, Severe acute respiratory syndrome coronavirus 2
YDEPTTTTSVPL

SEQ ID NO: 210 - protein, 12 aa, Severe acute respiratory syndrome coronavirus 2
SLVVRCSFYEDF

SEQ ID NO: 211 - protein, 12 aa, Severe acute respiratory syndrome coronavirus 2
SFYEDFLEYHDV

SEQ ID NO: 212 - protein, 12 aa, Severe acute respiratory syndrome coronavirus 2
LEYHDVRVVLDF

SEQ ID NO: 213 - protein, 12 aa, Severe acute respiratory syndrome coronavirus 2
EYHDVRVVLDFI

SEQ ID NO: 214 - protein, 6 aa, Severe acute respiratory syndrome coronavirus 2
FNDGVY

SEQ ID NO: 215 - protein, 12 aa, Severe acute respiratory syndrome coronavirus 2
CEFQFCNDPFLG

SEQ ID NO: 216 - protein, 45 aa, Severe acute respiratory syndrome coronavirus 2
KSWMESEFRVYSSANNCTFEYVSQPFLMDLEGKQGNFKNLREFVF

SEQ ID NO: 217 - protein, 16 aa, Severe acute respiratory syndrome coronavirus 2
SALEPLVDLPIGINIT

SEQ ID NO: 218 - protein, 21 aa, Severe acute respiratory syndrome coronavirus 2
KYNENGTITDAVDCALDPLSE

SEQ ID NO: 219 - protein, 7 aa, Severe acute respiratory syndrome coronavirus 2
NVYADSF

SEQ ID NO: 220 - protein, 5 aa, Severe acute respiratory syndrome coronavirus 2
PDDFT

SEQ ID NO: 221 - protein, 5 aa, Severe acute respiratory syndrome coronavirus 2
FERDI

SEQ ID NO: 222 - protein, 11 aa, Severe acute respiratory syndrome coronavirus 2
NGVEGFNCYFP

SEQ ID NO: 223 - protein, 37 aa, Severe acute respiratory syndrome coronavirus 2
GVLTESNKKFLPFQQFGRDIADTTDAVRDPQTLEILD

SEQ ID NO: 224 - protein, 18 aa, Severe acute respiratory syndrome coronavirus 2
PGTNTSNQVAVLYQDVNC

SEQ ID NO: 225 - protein, 16 aa, Severe acute respiratory syndrome coronavirus 2
IGAEHVNNSYECDIPI

SEQ ID NO: 226 - protein, 15 aa, Severe acute respiratory syndrome coronavirus 2
ALTGIAVEQDKNTQE

SEQ ID NO: 227 - protein, 19 aa, Severe acute respiratory syndrome coronavirus 2
KTPPIKDFGGFNFSQILPD

SEQ ID NO: 228 - protein, 12 aa, Severe acute respiratory syndrome coronavirus 2
SKRSFIEDLLFN

SEQ ID NO: 229 - protein, 9 aa, Severe acute respiratory syndrome coronavirus 2
QYGDCLGDI

SEQ ID NO: 230 - protein, 13 aa, Severe acute respiratory syndrome coronavirus 2
SRLDKVEAEVQID

SEQ ID NO: 231 - protein, 6 aa, Severe acute respiratory syndrome coronavirus 2
FPREGV

SEQ ID NO: 232 - protein, 23 aa, Severe acute respiratory syndrome coronavirus 2
NNTVYDPLQPELDSFKEELDKYF

SEQ ID NO: 233 - protein, 47 aa, Severe acute respiratory syndrome coronavirus 2
PDVDLGDISGINASVVNIQKEIDRLNEVAKNLNESLIDLQELGKYEQ

SEQ ID NO: 234 - protein, 24 aa, Severe acute respiratory syndrome coronavirus 2
CSCGSCCKFDEDDSEPVLKGVKLH

SEQ ID NO: 235 - protein, 33 aa, Severe acute respiratory syndrome coronavirus 2
TFGGPSDSTGSNQNGERSGARSKQRRPQGLPNN

SEQ ID NO: 236 - protein, 25 aa, Severe acute respiratory syndrome coronavirus 2
KFPRGQGVPINTNSSPDDQIGYYRR

SEQ ID NO: 237 - protein, 21 aa, Severe acute respiratory syndrome coronavirus 2
YLGTGPEAGLPYGANKDGIIW

SEQ ID NO: 238 - protein, 22 aa, Severe acute respiratory syndrome coronavirus 2
ANNAAIVLQLPQGTTLPKGFYA

SEQ ID NO: 239 - protein, 18 aa, Severe acute respiratory syndrome coronavirus 2
SQASSRSSSRSRNSSRNS

SEQ ID NO: 240 - protein, 43 aa, Severe acute respiratory syndrome coronavirus 2
LLLLDRLNQLESKRQKRTATKAYNVTQAFGRRGPEQTQGNFGD

SEQ ID NO: 241 - protein, 49 aa, Severe acute respiratory syndrome coronavirus 2
TVTKKSAAEASKKPRQKRTATKAYNVTQAFGRRGPEQTQGNFGDQELIR

SEQ ID NO: 242 - protein, 10 aa, Severe acute respiratory syndrome coronavirus 2
SAFFGMSRIG

SEQ ID NO: 243 - protein, 10 aa, Severe acute respiratory syndrome coronavirus 2
AIKLDDKDPN

SEQ ID NO: 244 - protein, 50 aa, Severe acute respiratory syndrome coronavirus 2
YKTFPPTEPKKDKKKKADETQALPQRQKKQQTVTLLPAADLDDFSKQLQQ

SEQ ID NO: 245 - protein, 13 aa, Severe acute respiratory syndrome coronavirus 2
MADSNGTITVEEL

SEQ ID NO: 246 - protein, 10 aa, Severe acute respiratory syndrome coronavirus 2
ILTRPLLESE

SEQ ID NO: 247 - protein, 15 aa, Severe acute respiratory syndrome coronavirus 2
LGRCDIKDLPKEITV

SEQ ID NO: 248 - protein, 10 aa, Severe acute respiratory syndrome coronavirus 2
AGDSGFAAYS

SEQ ID NO: 249 - protein, 17 aa, Severe acute respiratory syndrome coronavirus 2
MYSFVSEETGTLIVNSV

SEQ ID NO: 250 - protein, 38 aa, Severe acute respiratory syndrome coronavirus 2
VRGFGDSVEEVLSEARQHLKDGTCGLVEVEKGVLPQLE

SEQ ID NO: 251 - protein, 10 aa, Severe acute respiratory syndrome coronavirus 2
VEKGVLPQLE

SEQ ID NO: 252 - protein, 30 aa, Severe acute respiratory syndrome coronavirus 2
ARTAPHGHVMVELVAELEGIQYGRSGETLG

SEQ ID NO: 253 - protein, 10 aa, Severe acute respiratory syndrome coronavirus 2
SGETLGVLVP

SEQ ID NO: 254 - protein, 46 aa, Severe acute respiratory syndrome coronavirus 2
GGHSYGADLKSFDLGDELGTDPYEDFQENWNTKHSSGVTRELMREL

SEQ ID NO: 255 - protein, 18 aa, Severe acute respiratory syndrome coronavirus 2
RYVDNNFCGPDGYPLECI

SEQ ID NO: 256 - protein, 34 aa, Severe acute respiratory syndrome coronavirus 2
TKRGVYCCREHEHEIAWYTERSEKSYELQTPFEI

SEQ ID NO: 257 - protein, 10 aa, Severe acute respiratory syndrome coronavirus 2
DTFNGECPNF

SEQ ID NO: 258 - protein, 18 aa, Severe acute respiratory syndrome coronavirus 2
SEVGPEHSLAEYHNESGL

SEQ ID NO: 259 - protein, 18 aa, Severe acute respiratory syndrome coronavirus 2
TGVVGEGSEGLNDNLLEI

SEQ ID NO: 260 - protein, 18 aa, Severe acute respiratory syndrome coronavirus 2
NINIVGDFKLNEEIAIIL

SEQ ID NO: 261 - protein, 26 aa, Severe acute respiratory syndrome coronavirus 2
VYEKLKPVLDWLEEKFKEGVEFLRDG

SEQ ID NO: 262 - protein, 14 aa, Severe acute respiratory syndrome coronavirus 2
HSKGLYRKCVKSRE

SEQ ID NO: 263 - protein, 46 aa, Severe acute respiratory syndrome coronavirus 2
PKEIIFLEGETLPTEVLTEEVVLKTGDLQPLEQPTSEAVEAPLVGT

SEQ ID NO: 264 - protein, 30 aa, Severe acute respiratory syndrome coronavirus 2
PTKVTFGDDTVIEVQGYKSVNITFELDERI

SEQ ID NO: 265 - protein, 14 aa, Severe acute respiratory syndrome coronavirus 2
YTVELGTEVNEFAC

SEQ ID NO: 266 - protein, 30 aa, Severe acute respiratory syndrome coronavirus 2
QPVSELLTPLGIDLDEWSMATYYLFDESGE

SEQ ID NO: 267 - protein, 82 aa, Severe acute respiratory syndrome coronavirus 2
MYCSFYPPDEDEEEGDCEEEEFEPSTQYEYGTEDDYQGKPLEFGATSAALQPEEEQEEDWLDDDSQQTVGQQDGSEDNQTTT

SEQ ID NO: 268 - protein, 18 aa, Severe acute respiratory syndrome coronavirus 2
LEMELTPVVQTIEVNSFS

SEQ ID NO: 269 - protein, 18 aa, Severe acute respiratory syndrome coronavirus 2
DNVYIKNADIVEEAKKVK

SEQ ID NO: 270 - protein, 14 aa, Severe acute respiratory syndrome coronavirus 2
NNAMQVESDDYIAT

SEQ ID NO: 271 - protein, 42 aa, Severe acute respiratory syndrome coronavirus 2
VCVDTVRTNVYLAVFDKNLYDKLVSSFLEMKSEKQVEQKIAE

SEQ ID NO: 272 - protein, 54 aa, Severe acute respiratory syndrome coronavirus 2
KEEVKPFITESKPSVEQRKQDDKKIKACVEEVTTTLEETKFLTENLLLYIDING

SEQ ID NO: 273 - protein, 22 aa, Severe acute respiratory syndrome coronavirus 2
NYITTYPGQGLNGYTVEEAKTV

SEQ ID NO: 274 - protein, 38 aa, Severe acute respiratory syndrome coronavirus 2
KTTVASLINTLNDLNETLVTMPLGYVTHGLNLEEAARY

SEQ ID NO: 275 - protein, 30 aa, Severe acute respiratory syndrome coronavirus 2
TAYNGYLTSSSKTPEEHFIETISLAGSYKD

SEQ ID NO: 276 - protein, 6 aa, Severe acute respiratory syndrome coronavirus 2
GIEFLK

SEQ ID NO: 277 - protein, 30 aa, Severe acute respiratory syndrome coronavirus 2
KPHNSHEGKTFYVLPNDDTLRVEAFEYYHT

SEQ ID NO: 278 - protein, 22 aa, Severe acute respiratory syndrome coronavirus 2
GQQQTTLKGVEAVMYMGTLSYE

SEQ ID NO: 279 - protein, 34 aa, Severe acute respiratory syndrome coronavirus 2
LDGVVCTEIDPKLDNYYKKDNSYFTEQPIDLVPN

SEQ ID NO: 280 - protein, 6 aa, Severe acute respiratory syndrome coronavirus 2
KFADDL

SEQ ID NO: 281 - protein, 37 aa, Severe acute respiratory syndrome coronavirus 2
KVTFFPDLNGDVVAIDYKHYTPSFKKGAKLLHKPIVW

SEQ ID NO: 282 - protein, 31 aa, Severe acute respiratory syndrome coronavirus 2
SEEVVENPTIQKDVLECNVKTTEVVGDIILK

SEQ ID NO: 283 - protein, 10 aa, Severe acute respiratory syndrome coronavirus 2
AFGLVAEWFL

SEQ ID NO: 284 - protein, 17 aa, Severe acute respiratory syndrome coronavirus 2
DTFCAGSTFISDEVARD

SEQ ID NO: 285 - protein, 10 aa, Severe acute respiratory syndrome coronavirus 2
TDQSSYIVDS

SEQ ID NO: 286 - protein, 17 aa, Severe acute respiratory syndrome coronavirus 2
HSLSHFVNLDNLRANNT

SEQ ID NO: 287 - protein, 30 aa, Severe acute respiratory syndrome coronavirus 2
PILLLDQALVSDVGDSAEVAVKMFDAYVNT

SEQ ID NO: 288 - protein, 30 aa, Severe acute respiratory syndrome coronavirus 2
GFVDSDVETKDVVECLKLSHQSDIEVTGDS

SEQ ID NO: 289 - protein, 10 aa, Severe acute respiratory syndrome coronavirus 2
SEIIGYKAID

SEQ ID NO: 290 - protein, 10 aa, Severe acute respiratory syndrome coronavirus 2
ADFDTWFSQR

SEQ ID NO: 291 - protein, 9 aa, Severe acute respiratory syndrome coronavirus 2
YTPSKLIEY

SEQ ID NO: 292 - protein, 22 aa, Severe acute respiratory syndrome coronavirus 2
KPVPYCYDTNVLEGSVAYESLR

SEQ ID NO: 293 - protein, 23 aa, Severe acute respiratory syndrome coronavirus 2
SVRVVTTFDSEYCRHGTCERSEA

SEQ ID NO: 294 - protein, 22 aa, Severe acute respiratory syndrome coronavirus 2
RSLPGVFCGVDAVNLLTNMFTP

SEQ ID NO: 295 - protein, 14 aa, Severe acute respiratory syndrome coronavirus 2
FMRFRRAFGEYSHV

SEQ ID NO: 296 - protein, 18 aa, Severe acute respiratory syndrome coronavirus 2
NGVSFSTFEEAALCTFLL

SEQ ID NO: 297 - protein, 10 aa, Severe acute respiratory syndrome coronavirus 2
AMDTTSYREA

SEQ ID NO: 298 - protein, 10 aa, Severe acute respiratory syndrome coronavirus 2
GLWLDDVVYC

SEQ ID NO: 299 - protein, 11 aa, Severe acute respiratory syndrome coronavirus 2
DMLNPNYEDLL

SEQ ID NO: 300 - protein, 15 aa, Severe acute respiratory syndrome coronavirus 2
SCGSVGFNIDYDCVS

SEQ ID NO: 301 - protein, 18 aa, Severe acute respiratory syndrome coronavirus 2
TGVHAGTDLEGNFYGPFV

SEQ ID NO: 302 - protein, 17 aa, Severe acute respiratory syndrome coronavirus 2
AMKYNYEPLTQDHVDIL

SEQ ID NO: 303 - protein, 15 aa, Severe acute respiratory syndrome coronavirus 2
ILGSALLEDEFTPFD

SEQ ID NO: 304 - protein, 18 aa, Severe acute respiratory syndrome coronavirus 2
QSTQWSLFFFLYENAFLP

SEQ ID NO: 305 - protein, 10 aa, Severe acute respiratory syndrome coronavirus 2
LGVYDYLVST

SEQ ID NO: 306 - protein, 26 aa, Severe acute respiratory syndrome coronavirus 2
AVDINKLCEEMLDNRATLQAIASEFS

SEQ ID NO: 307 - protein, 10 aa, Severe acute respiratory syndrome coronavirus 2
AQEAYEQAVA

SEQ ID NO: 308 - protein, 14 aa, Severe acute respiratory syndrome coronavirus 2
VLKKLKKSLNVAKS

SEQ ID NO: 309 - protein, 14 aa, Severe acute respiratory syndrome coronavirus 2
ASALWEIQQVVDAD

SEQ ID NO: 310 - protein, 11 aa, Severe acute respiratory syndrome coronavirus 2
CTDDNALAYYN

SEQ ID NO: 311 - protein, 14 aa, Severe acute respiratory syndrome coronavirus 2
TIYTELEPPCRFVT

SEQ ID NO: 312 - protein, 14 aa, Severe acute respiratory syndrome coronavirus 2
AITVTPEANMDQES

SEQ ID NO: 313 - protein, 19 aa, Severe acute respiratory syndrome coronavirus 2
YGCSCDQLREPMLQSADAQ

SEQ ID NO: 314 - protein, 14 aa, Severe acute respiratory syndrome coronavirus 2
TDVVYRAFDIYNDK

SEQ ID NO: 315 - protein, 38 aa, Severe acute respiratory syndrome coronavirus 2
CCRFQEKDEDDNLIDSYFVVKRHTFSNYQHEETIYNLL

SEQ ID NO: 316 - protein, 14 aa, Severe acute respiratory syndrome coronavirus 2
TFSNYQHEETIYNL

SEQ ID NO: 317 - protein, 46 aa, Severe acute respiratory syndrome coronavirus 2
VYALRHFDEGNCDTLKEILVTYNCCDDDYFNKKDWYDFVENPDILR

SEQ ID NO: 318 - protein, 26 aa, Severe acute respiratory syndrome coronavirus 2
GIVGVLTLDNQDLNGNWYDFGDFIQT

SEQ ID NO: 319 - protein, 18 aa, Severe acute respiratory syndrome coronavirus 2
KYDFTEERLKLFDRYFKY

SEQ ID NO: 320 - protein, 14 aa, Severe acute respiratory syndrome coronavirus 2
QTYHPNCVNCLDDR

SEQ ID NO: 321 - protein, 18 aa, Severe acute respiratory syndrome coronavirus 2
FRELGVVHNQDVNLHSSR

SEQ ID NO: 322 - protein, 22 aa, Severe acute respiratory syndrome coronavirus 2
NKDFYDFAVSKGFFKEGSSVEL

SEQ ID NO: 323 - protein, 18 aa, Severe acute respiratory syndrome coronavirus 2
FFFAQDGNAAISDYDYYR

SEQ ID NO: 324 - protein, 25 aa, Severe acute respiratory syndrome coronavirus 2
QLLFVVEVVDKYFDCYDGGCINANQ

SEQ ID NO: 325 - protein, 14 aa, Severe acute respiratory syndrome coronavirus 2
YYDSMSYEDQDALF

SEQ ID NO: 326 - protein, 10 aa, Severe acute respiratory syndrome coronavirus 2
AATRGATVVI

SEQ ID NO: 327 - protein, 10 aa, Severe acute respiratory syndrome coronavirus 2
PHLMGWDYPK

SEQ ID NO: 328 - protein, 18 aa, Severe acute respiratory syndrome coronavirus 2
YECLYRNRDVDTDFVNEF

SEQ ID NO: 329 - protein, 6 aa, Severe acute respiratory syndrome coronavirus 2
MILSDD

SEQ ID NO: 330 - protein, 10 aa, Severe acute respiratory syndrome coronavirus 2
VKQGDDYVYL

SEQ ID NO: 331 - protein, 23 aa, Severe acute respiratory syndrome coronavirus 2
GAGCFVDDIVKTDGTLMIERFVS

SEQ ID NO: 332 - protein, 14 aa, Severe acute respiratory syndrome coronavirus 2
LTKHPNQEYADVFH

SEQ ID NO: 333 - protein, 18 aa, Severe acute respiratory syndrome coronavirus 2
TNDNTSRYWEPEFYEAMY

SEQ ID NO: 334 - protein, 9 aa, Severe acute respiratory syndrome coronavirus 2
RRPFLCCKC

SEQ ID NO: 335 - protein, 37 aa, Severe acute respiratory syndrome coronavirus 2
SDNVTDFNAIATCDWTNAGDYILANTCTERLKLFAAE

SEQ ID NO: 336 - protein, 29 aa, Severe acute respiratory syndrome coronavirus 2
TEETFKLSYGIATVREVLSDRELHLSWEV

SEQ ID NO: 337 - protein, 11 aa, Severe acute respiratory syndrome coronavirus 2
IGEYTFEKGDY

SEQ ID NO: 338 - protein, 20 aa, Severe acute respiratory syndrome coronavirus 2
QEHYVRITGLYPTLNISDEF

SEQ ID NO: 339 - protein, 21 aa, Severe acute respiratory syndrome coronavirus 2
FCTVNALPETTADIVVFDEIS

SEQ ID NO: 340 - protein, 17 aa, Severe acute respiratory syndrome coronavirus 2
PRTLLTKGTLEPEYFNS

SEQ ID NO: 341 - protein, 18 aa, Severe acute respiratory syndrome coronavirus 2
TCRRCPAEIVDTVSALVY

SEQ ID NO: 342 - protein, 10 aa, Severe acute respiratory syndrome coronavirus 2
QIGVVREFLT

SEQ ID NO: 343 - protein, 14 aa, Severe acute respiratory syndrome coronavirus 2
TVDSSQGSEYDYVI

SEQ ID NO: 344 - protein, 14 aa, Severe acute respiratory syndrome coronavirus 2
MSDRDLYDKLQFTS

SEQ ID NO: 345 - protein, 38 aa, Severe acute respiratory syndrome coronavirus 2
NYQVNGYPNMFITREEAIRHVRAWIGFDVEGCHATREA

SEQ ID NO: 346 - protein, 15 aa, Severe acute respiratory syndrome coronavirus 2
TGYVDTPNNTDFSRV

SEQ ID NO: 347 - protein, 18 aa, Severe acute respiratory syndrome coronavirus 2
WHHSIGFDYVYNPFMIDV

SEQ ID NO: 348 - protein, 14 aa, Severe acute respiratory syndrome coronavirus 2
DWTIEYPIIGDELK

SEQ ID NO: 349 - protein, 6 aa, Severe acute respiratory syndrome coronavirus 2
ADKFPV

SEQ ID NO: 350 - protein, 9 aa, Severe acute respiratory syndrome coronavirus 2
QADVEWKFY

SEQ ID NO: 351 - protein, 35 aa, Severe acute respiratory syndrome coronavirus 2
KAYKIEELFYSYATHSDKFTDGVCLFWNCNVDRYP

SEQ ID NO: 352 - protein, 10 aa, Severe acute respiratory syndrome coronavirus 2
VCRHHANEYR

SEQ ID NO: 353 - protein, 18 aa, Severe acute respiratory syndrome coronavirus 2
KGHFDGQQGEVPVSIINN

SEQ ID NO: 354 - protein, 21 aa, Severe acute respiratory syndrome coronavirus 2
YTKVDGVDVELFENKTTLPVN

SEQ ID NO: 355 - protein, 27 aa, Severe acute respiratory syndrome coronavirus 2
IKPVPEVKILNNLGVDIAANTVIWDYK

SEQ ID NO: 356 - protein, 9 aa, Severe acute respiratory syndrome coronavirus 2
ANTVIWDYK

SEQ ID NO: 357 - protein, 22 aa, Severe acute respiratory syndrome coronavirus 2
TETICAPLTVFFDGRVDGQVDL

SEQ ID NO: 358 - protein, 38 aa, Severe acute respiratory syndrome coronavirus 2
KPRSQMEIDFLELAMDEFIERYKLEGYAFEHIVYGDFS

SEQ ID NO: 359 - protein, 22 aa, Severe acute respiratory syndrome coronavirus 2
IGLAKRFKESPFELEDFIPMDS

SEQ ID NO: 360 - protein, 15 aa, Severe acute respiratory syndrome coronavirus 2
VCSVIDLLLDDFVEI

SEQ ID NO: 361 - protein, 10 aa, Severe acute respiratory syndrome coronavirus 2
DLQNYGDSAT

SEQ ID NO: 362 - protein, 38 aa, Severe acute respiratory syndrome coronavirus 2
LLVDSDLNDFVSDADSTLIGDCATVHTANKWDLIISDM

SEQ ID NO: 363 - protein, 42 aa, Severe acute respiratory syndrome coronavirus 2
WTAFVTNVNASSSEAFLIGCNYLGKPREQIDGYVMHANYIFW

SEQ ID NO: 364 - protein, 14 aa, Severe acute respiratory syndrome coronavirus 2
NYLGKPREQIDGYV

SEQ ID NO: 365 - protein, 22 aa, Severe acute respiratory syndrome coronavirus 2
ACHNSEVGPEHSLAEYHNESGL

SEQ ID NO: 366 - protein, 34 aa, Severe acute respiratory syndrome coronavirus 2
QRKQDDKKIKACVEEVTTTLEETKFLTENLLLYI

SEQ ID NO: 367 - protein, 15 aa, Severe acute respiratory syndrome coronavirus 2
YGCSCDQLREPMLQS

SEQ ID NO: 368 - protein, 18 aa, Severe acute respiratory syndrome coronavirus 2
LYDANYFLCWHTNCYDYC

SEQ ID NO: 369 - protein, 14 aa, Severe acute respiratory syndrome coronavirus 2
GDGTTSPISEHDYQ

SEQ ID NO: 370 - protein, 52 aa, Severe acute respiratory syndrome coronavirus 2
GVEHVTFFIYNKIVDEPEEHVQIHTIDGSSGVVNPVMEPIYDEPTTTTSVPL

SEQ ID NO: 371 - protein, 14 aa, Severe acute respiratory syndrome coronavirus 2
ALITLATCELYHYQ

SEQ ID NO: 372 - protein, 14 aa, Severe acute respiratory syndrome coronavirus 2
SSGTYEGNSPFHPL

SEQ ID NO: 373 - protein, 18 aa, Severe acute respiratory syndrome coronavirus 2
PKLFIRQEEVQELYSPIF

SEQ ID NO: 374 - protein, 13 aa, Severe acute respiratory syndrome coronavirus 2
MIELSLIDFYLCF

SEQ ID NO: 375 - protein, 12 aa, Severe acute respiratory syndrome coronavirus 2
LELQDHNETCHA

SEQ ID NO: 376 - protein, 10 aa, Severe acute respiratory syndrome coronavirus 2
HQPYVVDDPC

SEQ ID NO: 377 - protein, 7 aa, Severe acute respiratory syndrome coronavirus 2
VVNFNLT

(19) United States
(12) Patent Application Publication
(10) Pub. No.: US 2024/0192211 A1
(43) Pub. Date: Jun. 13, 2024

(54) PEPTIDES AND THEIR USE IN DIAGNOSIS OF SARS-COV-2 INFECTION

(71) Applicants: Biotome Pty Ltd, Nedlands Western Australia (AU); Vivocens AB, Gothenburg (SE)

(72) Inventors: Bror Samuel LUNDIN, North Fremantle Western Australia (AU); Ali Mohaghegh HARANDI, Gothenburg (SE); Alma FULURIJA, North Fremantle Western Australia (AU)

(73) Assignees: Biotome Pty Ltd, Nedlands Western Australia (AU); Vivocens AB, Gothenburg (SE)

(21) Appl. No.: 18/278,647

(22) PCT Filed: Feb. 24, 2022
(86) PCT No.: PCT/AU2022/050153

§ 371 Date: Aug. 24, 2023

(30) Foreign Application Priority Data
Feb. 24, 2021 (AU) 2021900506

(51) Int. Cl. G01N 33/569; C07K 14/005

(52) U.S. Cl. CPC G01N 33/56983; C07K 14/005; C12N 2770/20022; G01N 2333/165; G01N 2469/20

(57) ABSTRACT

Uses and methods for diagnosing a SARS-COV-2 infection in a subject or the detection of the presence of SARS-COV-2 in a subject are provided, and which include the step of assaying a sample from the subject for the presence of antibodies that specifically bind to at least one peptide sequence derived from a linear epitope of any one or more of the S, N, or ORF1 proteins, or combinations thereof, of SARS-COV-2.

### FIELD OF THE INVENTION

This invention relates to peptides from the SARS-COV-2 virus. The peptides of the invention can be used for diagnosis of SARS-COV-2 infection in a subject.

### BACKGROUND OF THE INVENTION

The following discussion of the background art is intended to facilitate an understanding of the present invention only. The discussion is not an acknowledgement or admission that any of the material referred to is or was part of the common general knowledge as at the priority date of the application.

Coronavirus disease 2019 (COVID-19) is a contagious disease caused by a severe acute respiratory syndrome coronavirus, termed SARS-COV-2. Symptoms of COVID-19 are variable, but often include fever, cough, fatigue, dyspnoea, and loss of smell and taste. Symptoms begin one to fourteen days after exposure to the virus. Currently, of those who develop noticeable symptoms, most (81%) develop mild to moderate symptoms (up to mild pneumonia), while 14% develop severe symptoms (dyspnoea, hypoxia, or more than 50% lung involvement on imaging), and 5% suffer critical symptoms (respiratory failure, shock, or multiorgan dysfunction. At least a third of the people who are infected with the virus remain asymptomatic and do not develop noticeable symptoms at any point in time, but they still can spread the disease. Some people continue to experience a range of effects—known as “long COVID”—for months after recovery, and damage to organs has been observed. The COVID-19 pandemic has illustrated the need for serology diagnostics with improved accuracy for detecting not only SARS-COV-2 infection, but also different strains thereof. Given that many coronavirus strains and sub-types other than SARS-COV-2 share antigens with SARS-COV-2, there is significant risk of false positives using existing antibody diagnostics of which the Applicant is aware. It is therefore an object of this invention to address some of the shortcomings of prior detection systems for diagnosing or confirming SARS-COV-2 infection by way of an antibody test.

### SUMMARY OF THE INVENTION

Broadly, the invention relates to peptides comprising linear epitopes from SARS-COV-2 that find use in diagnostic applications related to SARS-COV-2-associated diseases including, specifically, identification of subjects at risk of developing COVID-19 and pathologies relating to SARS-COV-2 infection.

The term “linear epitope” or a “sequential epitope” as used herein is an epitope that is recognised by antibodies by its linear sequence of amino acids, or primary structure. In contrast, most antibodies recognise a conformational epitope that has a specific three-dimensional shape and its protein structure. This has implications for increased sensitivity and specificity when constructing immunological tests or assays, by making use of the peptides of the present invention to identify subjects infected with SARS-COV-2, especially against a background of antibodies generated against other, prior human coronavirus infections, specifically but not limited to endemic seasonal coronaviruses that may cause false positive tests.

From all peptides present in the proteome of SARS-COV-2, the Applicant has defined a subset that is recognised by antibodies from humans infected with SARS-COV-2. In general, a significant number of SARS-COV-2 peptides will react with serum from non-infected patients or individuals previously infected with other coronaviruses, i.e. the vast majority of adults. Within the subset of peptides recognised by antibodies, the Applicant has identified the smaller subset of peptides that has a diagnostic capacity; and finally, in this subset of diagnostic peptides, the Applicant has identified the crucial amino acid sequence(s) having the highest diagnostic capacity. In other words, the diagnostic capacity does not stem from only the presence/absence of antibodies binding to these peptides in the infected individual, but crucially also from only a small subset of these peptides associated with an antibody-response that is present in SARS-COV-2 infected individuals but that is absent in non-infected individuals.

According to one aspect of the invention, there is provided use of at least one peptide sequence derived from a linear epitope of the SARS-COV-2 virus, for the identification of subjects infected with SARS-COV-2.

The at least one peptide may be derived from a linear epitope of one or more of the S, N, or ORF1 proteins, or various combinations thereof, for the identification of subjects infected with SARS-COV-2.

As such, the invention extends to a peptide comprising or consisting of an amino acid sequence selected from the group consisting of SEQ ID NO 1-22, in particular any one or more of the amino acid sequences selected from the group consist of SEQ ID NO 1-5.

Furthermore, the invention extends to a method of diagnosing COVID-19 in a subject, the method including the step of assaying a sample from the subject for the presence of at least one peptide sequence derived from a linear epitope of any one or more of the S, N, or ORF1 proteins, or various combinations thereof, of SARS-COV-2. This may include assaying for the presence of one or more linear epitopes in the same SARS-COV-2 protein, i.e. combining peptides containing amino acid sequences of linear epitopes within the same protein. This may also include assaying for the presence of one or more linear epitopes in different SARS-CoV-2 proteins. The linear epitope from the ORF1 protein may be from the ORF1ab protein.

According to one aspect of the invention, there is provided a method of diagnosing a SARS-CoV-2 infection in a subject, the method including the step of assaying a sample from the subject for the presence of any one or more of the following epitopes, including various combinations thereof:
in protein S, peptides within epitopes S_005, S_010, S_019 and S_021; viz. SEQ ID NO 2, 4, 6, 7, 11, 13, 15 and 18;
in protein N, peptides within epitopes N_006 and N_010; viz. SEQ ID NO 1, 3, 5, 12 and 19; and
in the ORF1ab polyprotein, peptides within epitopes ORF1a_005, ORF1a_018 and ORF1a_068; viz. SEQ ID NO 8, 9, 10, 14, 16 and 17.

The above peptides of the invention find application especially when assaying for discriminating IgG antibody and its subclasses levels. For IgA responses, the IgA-discriminatory peptides of the invention belonged to S_005, S_010, S_021, N_010, ORF1a_018, 068 and ORF1a_090 viz. SEQ ID NO 1, 2, 3, 10, 13, 15, 16, 20, 21 and 22.

The invention extends to a diagnostic combination of 3 or more peptides of the invention. As such, the invention extends to diagnostic 3-peptide combinations for IgG-antibodies comprising any one or more of the combinations selected from the group consisting of:
SEQ ID NO 1 in combination with SEQ ID NO 2 and any one of SEQ ID NOS 7, 15, 18, 31, 35, 67, 113 and 139; and
SEQ ID NOS 2, 74 and 128.

Furthermore, the invention extends to diagnostic 3-peptide combinations for IgA-antibodies, which may include are any or more of the following combinations:
(i) SEQ ID NO 2 in combination with SEQ ID NO 15 and any one of SEQ ID NOS 1 or 159;
(ii) SEQ ID NO 2 in combination with SEQ ID NOs 22 and 40;
(iii) SEQ ID NO 2 in combination with SEQ ID NOs 22 and 128;
(iv) SEQ ID NO 2 in combination with SEQ ID NOs 13 and 143;
(v) SEQ ID NO 2 in combination with SEQ ID NOs 30 and 140.

The method of diagnosing SARS-COV-2 infection may comprise the steps of:
(i) providing a biopsy sample containing antibodies of the subject;
(ii) bringing the sample into contact with any one or more of the peptides of the invention; and
(iii) detecting the binding of the antibodies with any one or more peptides of the invention.

Step (iii) may also include detecting the binding of the antibodies using any of the combination of 2 or 3 peptide combinations, set out hereinbefore.

The sample may include, but need not be limited to, bodily fluid samples containing antibodies, such as a whole blood, serum, plasma, saliva, tear fluid, broncho-alveolar fluid, buccal brush extract or a tissue sample.

By “discriminatory” is meant peptides that are recognized by antibodies from SARS-COV-2-infected individuals with minimal cross-reactivity to other coronaviruses or to other viruses or pathogens.

Preferably, said peptide sequence comprises at most 25 amino acids, more preferably 20 or 21 amino acids. In some cases, said peptide sequence comprises 15 amino acids or fewer, even as few as 12 amino acids, or even as few as 10, 9, 8, or 7 amino acids, while still retaining the ability to serve as discriminatory linear peptides for detecting SAR-COV-2 infections.

The peptide or peptides of the invention may be a non-naturally occurring peptide or peptides, and may be modified.

The peptides of the invention have the advantage that they can be used for identification, confirmation or diagnosis of SARS-COV-2 infection and COVID-19-associated diseases. The Applicant believes that diagnosis of subjects presently infected by, or previously infected by, SARS-COV-2 using the peptides of the invention results in far fewer false positives, if any, than existing antibody diagnostic assays and commercially available kits of which the Applicant is aware, especially for SARS-COV-2 of the original Wuhan strain and of the key new SARS-COV-2-variants, including B.1.1.7 and B.1.351.

Given that the peptides of the present invention have been designed to have optimal discriminatory propensities, the Applicant is of the opinion that there is no measurable, or significantly lower, background binding of antibodies to the peptides in individuals not currently and not previously infected by SARS-COV-2. Advantageously, the peptides of the invention are short and can therefore be manufactured at large scale and at low cost. A further advantage includes the inherent chemistry of linear peptides of the present invention that makes them amenable to adding tags for linkage to different solid phases for various state-of-the-art antibody assays.

In a further aspect of the invention there is provided a diagnostic assay or a diagnostic kit comprising a peptide according to one aspect of the invention or a mixture of peptides according to the invention. The assay or kit is preferably an assay or kit for diagnosis, more specifically diagnosis of SARS-COV-2 infection. The assay or kit may include a microarray chip including one or more peptides of the invention, and the assay or kit may include an Enzyme Linked Immunosorbent Assay (ELISA), a multiplex bead-based antibody assay, a non-labelling antigen-antibody detection assay (such as a surface plasmon resonance assay, a Bio Layer Interferometry assay), a lateral flow assay or an electrochemical biosensor including, but not limited to, a graphene-based field-effect transistor.

In another aspect of the invention there is provided a mixture of at least two peptides of the invention. Such a mixture has the advantage that it can be used for detecting two or more different SARS-COV-2 strains in a subject. The mixtures can be used in the same manner as the peptides herein.

### DETAILED DESCRIPTION OF THE INVENTION

The following embodiments, given by way of non-limiting example only, are described in order to provide a more precise understanding of the subject matter of a preferred embodiment or embodiments. This description is included solely for the purposes of exemplifying the present invention. It should not be understood as a restriction on the broad summary, disclosure or description of the invention as set out above.

The Applicant is pursuing a precision immunology concept by focusing on linear B-cell epitopes in the form of short peptides for use in diagnosing SARS-COV-2 infections with higher accuracy than conventional serological or immunological diagnostics. Linear epitopes are not always suitable for analysis of antibody functions, but unlike conformational B-cell epitopes, the Applicant has invented methods suitable for detection of linear B-cell epitopes useful for precision diagnosis of SARS-COV-2. In addition, having optimised the size of peptides used for analysis to identify linear B-cell epitopes for use in the invention, the low cost of synthesis of peptides once the appropriate peptides have been identified make them ideal candidates as the basis for precision immunology diagnostics, especially for multiplex tests where several combinations of peptides may be used.

The aim of this study was to harness the Applicant's precision immunology invention to identify linear B-cell epitopes of SARS-COV-2 that may be used to develop more accurate and specific antibody diagnostics for such infections. The Applicant developed and used peptides, functional peptide fragments (i.e. minimally sized epitopes that can still function to diagnose SARS-COV-2 infection), and peptide array technology to test the capacity of serum antibodies to bind previously well-defined proteins of the SARS-COV-2 proteome. The Applicant has, in their opinion, invented useful, differentially discriminatory linear B cell epitopes, and sets of such epitopes, of SARS-COV-2 that find use for precision antibody diagnosis of SARS-COV-2 infection.

By utilising high-precision serology, with resolution at the peptide level, specifically at the level of linear epitopes (instead of at the broader protein level or conformational B-cell epitopes), the Applicant has now identified peptides containing highly specific linear epitopes to which there is a strong antibody-response only in individuals currently or previously infected with SARS-COV-2. These sequences are thus indicative of SARS-COV-2 infection as compared to other human coronavirus subtypes. Therefore, the diagnostic peptides containing linear epitopes that the Applicant has identified are predicted to have both high sensitivity and specificity as determined by receiver operator characteristic area under curve (ROC AUC) values, and are useful for diagnostic applications and address some of the shortcomings of the currents tests of which the Applicant is aware.

Reference is made herein to an interval of sequences. This refers to all the sequences in the interval, thus for example “SEQ ID NO 2 to SEQ ID NO 5” refers, inclusively, to SEQ ID NO, 2, 3, 4, and 5. Sequences are written using the standard one-letter annotation for amino acid residues. The amino acid residues are preferably connected with peptide bonds but may, in certain instances, be connected with alternative bonds known to those skilled in the field of the invention.

Some peptides herein may have sequence variability. Thus, certain sequences may specify a position in the sequence that can be any amino acid. This may be indicated with an X or, in the sequence listing, Xaa. The X or Xaa can be replaced with any amino acid, preferably any L-amino acid, including amino acids resulting from post translational modification, such as citrulline. The amino acid does not have to be a naturally occurring amino acid. Preferably the amino acid does not have a bulky side chain, as a bulky side chain could prevent antibody binding. A suitable molecular weight of the amino acid may be from 85 D to 300 D, more preferably from 89 D to 220 D.

In general, the peptide may comprise or consist of an amino acid or peptide sequence selected from the group consisting of SEQ ID NO 1 to SEQ ID NO 22 (Table 3). Specifically, SEQ ID NO 1 to 5 are the most highly discriminatory and form an important part of the invention and may be used individually for diagnosis. They can also be used in combination together with other SARS-COV-2 linear epitope sequences described herein for diagnostic purposes.

The peptides of the invention may comprise parts or functional fragments of the sequences of SEQ ID NO 1 to SEQ ID NO 22 to which antibodies can be generated that can be used for the positive identification of SARS-COV-2 infection.

In certain embodiments, the amino acid may be replaced in a conserved manner, wherein, for example, a hydrophobic amino acid is replaced with a different hydrophobic amino acid, or where a polar amino acid is replaced with a different polar amino acid.

The invention also extends to combinations of such peptides for use in identification or diagnosis of SARS-COV-2 infection.

In one embodiment of the invention, a peptide comprising or consisting of any one of SEQ ID NO 1 to 22 is used. These sequences comprise the minimal binding regions of certain antibodies that find use in the present invention. These peptides have the advantage that the diagnostic accuracy is higher than conventional tests of which the Applicant is aware, since they are predicted to elicit a strong, highly selective antibody-response in a high percentage of individuals carrying a SARS-COV-2 infection.

Preferably, said peptide sequence comprises at most 25 amino acids, more preferably 15 amino acids, even more preferably, at most 12 or even 11 amino acids. Shorter peptides may be desirable because it results in less unspecific binding (by an antibody) and therefore less background, and peptides as short as 10, 9, 8, or even 7 amino acids find application in the present invention. However, peptides that are too short may not be discriminatory. However, a longer peptide may in some cases be desirable to allow for exposing the linear epitope to allow antibody binding without steric hindrance.

Preferably the peptide binds specifically (in the immunological sense) and with high affinity to an antibody, preferably an antibody from a subject sample that also binds to linear epitopes of the SARS-COV-2 S, N, and ORF1a proteins, although in certain embodiments use can be made of peptides that bind with low affinity to an antibody and still find use in diagnosis. An antibody-peptide interaction is said to exhibit “specific binding” or “preferential binding” in the immunological sense if it reacts or associates more frequently, more rapidly, with greater duration and/or with greater affinity with a particular cell or substance than it does with alternative cells or substances. An antibody “specifically binds” or “preferentially binds” to a peptide if it binds with greater affinity, avidity, more readily, and/or with greater duration than it binds to other substances. Binding can be determined with any suitable method. Binding can be determined by methods known in the art, for example ELISA, surface plasmon resonance, Bio Layer Interferometry, Western blot or the other methods described herein (see below). Such methods can be used by those skilled in the art to determine suitable lengths or amino acid sequences of the peptide.

Preferably the use of the peptide has both a high diagnostic specificity and a high diagnostic sensitivity. In any diagnostic test, these two properties are dependent on what level is used as the cut-off for a positive test. To assess diagnostic accuracy independently of a set cut-off, a receiver operator characteristic curve (ROC curve) can be used. In an ROC curve, true positive rate (sensitivity) is plotted against false positive rate (1-specificity) as the cut-off is varied from 0 to infinity. The area under the ROC curve (ROC AUC) is then used to estimate the overall diagnostic accuracy. Preferably the use of the peptide has an ROC AUC of at least 0.55, for example an ROC AUC of at least, 0.60, 0.65, 0.70, 0.75, 0.80, 0.85, 0.90, 0.95, 0.96, 0.97, 0.98, 0.99 or an ROC AUC of 1.00. Preferably, the use of the peptide has ROC AUC of at least 0.85, and most preferably an ROC AUC of 1 or close to 1.

As used herein, the term “peptide” is used to mean peptides, fragments of proteins and the like, including peptidomimetic compounds. The term “peptidomimetic”, means a peptide-like molecule that has the activity of the peptide upon which it is structurally based, the activity being specific and high affinity binding to antibodies that bind to linear epitopes of the SARS CoV-2 proteins. Such peptidomimetics include chemically modified peptides, peptide-like molecules containing non-naturally occurring amino acids (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861). A variety of peptidomimetics are known in the art including, for example, peptide-like molecules which contain a constrained amino acid. In certain embodiments circular peptides may be used.

The term “functional fragment” as used herein refers to truncated forms of SEQ ID NO 1 to 19 which consist of contiguous amino acid sequences identical to contiguous amino acid sequences of such sequences and which are capable of being used in the methods of the invention to identify subjects infected, or previously infected, with SARS-COV-2.

As mentioned hereinbefore, the term “linear epitope” or a “sequential epitope” as used herein is an epitope that is recognised by antibodies by its linear sequence of amino acids, or primary structure.

The peptide may be an isolated peptide meaning a peptide in a form other than it occurs in nature, e.g. in a buffer, in a dry form awaiting reconstitution, as part of a kit, and the like.

The invention further extends to any protein product of the S, N, or ORF1a genes which include a peptide of SEQ ID NOS 1 to 22.

The peptide may be substantially purified or isolated, meaning a peptide that is devoid of unintended amino acids, and substantially free of proteins, lipids, carbohydrates, nucleic acids and other biological materials with which it is naturally associated. For example, a substantially pure peptide can be at least about 60% of dry weight, preferably at least about 70%, 80%, 90%, 95%, or 99% of dry weight.

A peptide of the present invention can be in the form of a salt. Suitable acids and bases that are capable of forming salts with the peptides are well known to those of skill in the art, and include inorganic and organic acids and bases, including potassium, calcium, magnesium, or sodium salts. The peptide can be provided in a solution, for example an aqueous solution. Such a solution may comprise suitable buffers, salts, protease inhibitors, or other suitable components as is known in the art.

The peptide can, in certain embodiments of the invention, be associated with (e.g. coupled, fused or linked to, directly or indirectly) one or more additional moieties as is known in the art. Non-limiting examples of such moieties include peptide or non-peptide molecules such as biotin, a poly-his tag, GST, a FLAG-tag, or a linker or a spacer. The association may be a covalent or non-covalent bond. The association may be, for example, via a terminal cysteine residue or a chemically reactive linking agent, the biotin-avidin system or a poly-his tag. For example, the peptide may be linked with a peptide bond to a single biotin-conjugated lysine residue, in which the lysine is biotinylated via the epsilon amino groups on its side chain, such as the peptide example H-XXXXXXXXXXXXXXX(K(Biotin))-NH2, where X indicates the amino acids of the peptide.

The associated moiety may be used to attach or link the peptide, to improve purification, to enhance expression of the peptide in a host cell, to aid in detection, to stabilise the peptide, and the like. In the case of a short peptide attached to a substrate, for example a solid phase, it may be desirable to use a linker or a spacer to ensure exposure of the peptide to antibodies so that the antibodies can bind.

The peptide may be associated with a substrate that immobilises the peptide. The substrate may be, for example, a solid or semi-solid carrier, a solid phase, support or surface. The peptide may be immobilised on a solid support or be present in a liquid. Examples includes beads or wells in plates, such as microtiter plates, such as 96-well plates, and also include surfaces of lab-on-a-chip diagnostic or similar devices. The association can be covalent or non-covalent and can be facilitated by a moiety associated with the peptide that enables covalent or non-covalent binding, such as a moiety that has a high affinity to a component attached to the carrier, solid phase, support or surface. For example, the biotin-avidin system can be used.

The peptides of the present invention find application in detecting SARS-COV-2-specific linear epitope antibodies in a sample from a subject, the method comprising contacting a biological sample with a peptide as described herein and detecting binding of antibodies in the sample to the peptide to infer whether the subject has, or had, a SARS-COV-2 infection. The peptide may be associated with a substrate that immobilises the peptide, as described herein, for example attached to a solid support. The method may include incubation to allow binding, washing, and detection of antibodies as described herein. Methods for detecting binding of antibodies are described below and include, for example, immunoblotting, ELISA, or Western blot.

The peptides can be used for diagnosis and/or prognosis, in particular for identifying SARS-CoV-2 strains predisposed to resulting in greater or lesser levels of pathology in subjects.

The present invention further relates to the use of the described methods and kits for the diagnosis, prognosis and risk assessment of SARS-COV-2 in human or animal subjects.

The term “sample” as used herein refers to a bodily fluid sample obtained for the purpose of diagnosis, prognosis or evaluation of a subject in question, e.g., a patient. Preferred test samples include blood, serum, plasma, cerebrospinal fluid, urine, saliva and pleural effusion. In addition, those skilled in the art will appreciate that some test samples are easily analysed according to fractionation or purification means, such as separation of whole blood into serum or plasma components. In one embodiment, the sample is preferably a blood sample.

Thus, in a preferred embodiment of the invention, the sample is selected from the group consisting of a blood sample, a serum sample, a plasma sample, a cerebrospinal fluid sample, a saliva sample, and a urine sample or any extract of said sample. Preferably, the sample is a blood sample, most preferably a serum sample or a plasma sample. The sample may also be a tissue sample or may be derived from a harvesting procedure, such as during a gastroscopy.

Identification, diagnosis, or prognosis can be carried out using any suitable method. In a preferred method, antibodies in a sample from a subject are allowed to bind to one or more peptides of the invention, and binding is detected using detection methods known in the art. The subject can be a human or an animal, preferably a human. Binding in vitro of antibodies from the subject to one or more peptides of the invention indicates that the immune system of the subject has generated antibodies against that particular peptide and thus that said at least one peptide and hence that linear epitopes of SARS-COV-2 of the present invention are associated with increased risk of pathology present in the subject.

The method, in one embodiment, thus comprises the steps of (1) isolating, from a subject, a sample of body fluid or tissue likely to contain antibodies or providing, in vitro, such a sample; (2) contacting the sample with a peptide, under conditions effective for the formation of a specific peptide-antibody complex (for specific binding of the peptide to the antibody), e.g., reacting or incubating the sample and a peptide; and (3) assaying the contacted (reacted) sample for the presence of an antibody-peptide reaction (for example determining the amount of an antibody-peptide complex). The method may involve one or more washing steps, as is known in the art. Steps 2 and 3 are preferably carried out in vitro, that is, using the sample after the sample has been isolated from the subject, in a sample previously isolated from a subject, but can also be carried out in a different environment.

Antibody-response to the peptides can be detected by different immunological/serological methods. Suitable formats of detecting presence of the antibody using the peptides includes peptide micro arrays, lateral flow assays, ELISA, non-labelling antigen-antibody assays such as surface plasmon resonance and Biolayer Interferometry assays, chromatography, Western blot, lab-on-a-chip formats, microbead-based or planar single- or multiplex immunoassays, microelectromechanical systems (MEMS), electrochemical biosensors, field-effect transistors and the like.

Often these methods involve proving the peptide bound to stationary phase (such as the well of an ELISA plate or the surface of a microbead) and adding the sample to be analysed in the liquid phase, allowing antibodies to bind and then washing away unbound antibodies. Antibody binding can be detected in vitro by using a labelled secondary antibody that binds to a specific type of human antibody for example IgG, IgA, IgG1, IgG2, IgG3, or IgG4. In ELISA, the secondary antibody is labelled with an enzyme, such as horseradish peroxidase (HRP) or alkaline phosphatase (AP). The secondary antibody is suitably from another species than human, for example from rabbit or goat. Alternatively, a fluorescence label or radioactive label can be used.

A protocol for using the peptides in an ELISA can be easily optimised by a person skilled in the art with regard to which secondary antibody to use, its dilution, buffers, blocking solution, wash and the like. An outline of an example of an ELISA protocol using plates can be as follows: Polystyrene microtiter plates are coated with optimal concentrations, as determined by checkerboard titrations, of the peptides of interest dissolved in PBS at room temperature overnight. After two washes with PBS, wells are blocked with 0.1% (wt/vol) bovine serum albumin-PBS at 37° C. for 30 min. Subsequent incubations are performed at room temperature, and plates are washed three times with PBS containing 0.05% Tween (PBS-Tween) between incubations. Samples of serum or other bodily fluids are added in duplicates or triplicates in initial dilutions of for example 1/10 and diluted for example in a three-fold dilution series. Control samples previously tested and found to have antibodies to the peptides were used as positive controls. Samples with known concentrations of antibodies may be used for creating a standard curve. Wells to which only PBS-Tween® are added are used as negative controls for determination of background values. After incubation at room temperature for 90 min, HRP-labeled rabbit anti-human IgA or IgG antibodies are added and incubated for 60 min. Plates are thereafter read in a spectrophotometer 20 min after addition of H₂O₂ and ortho-phenylene-diamine dihydrochloride in 0.1 M sodium citrate buffer, pH 4.5. The end point titers of each sample are determined as the reciprocal interpolated dilution giving an absorbance of for example 0.4 above background at 450 nm. Alternatively, as the final read-out value, the absorbance value can be used. The skilled person recognises that this ELISA protocol is an example only and many different variants and alterations of this protocol are possible.

Alternatively, in one embodiment, B-cells are isolated from the subject, and it is analysed if the cells are able to produce antibodies that bind to the peptide. This can be done by using the ELISPOT method, ALS (antibodies in lymphocyte secretions), or similar methods.

Diagnosis can also be carried out by detecting the presence of linear epitopes of SARS-COV-2 proteins assayed for in the present invention in a tissue sample from a patient using antibodies specific for a peptide selected from peptides comprising or consisting of SEQ ID NO 1-22, more particularly SEQ ID NO 1-8, and combinations thereof.

Antibodies with the desired binding specificity can be generated by a person skilled in the art. The antibody can be a polyclonal or a monoclonal antibody, with monoclonal antibodies being preferred. The antibody can be used in any useful format to detect the proteins or peptides, for example Western blot, ELISA, immunohistochemistry, and the like. The antibody can be used for the diagnostic methods herein.

The peptides can be synthesised by methods known in the art. The peptides can be obtained substantially pure and in large quantities by means of organic synthesis, such as solid phase synthesis. Methods for peptide synthesis are well known in the art, for example using a peptide synthesis machine. Of course, the peptides may be ordered from a peptide synthesis company.

The peptides can also be of animal, plant, bacterial or virus origin. The peptide may then be purified from the organism, as is known in the art. The peptide can be produced using recombinant technology, for example using eukaryotic cells, bacterial cells, or virus expression systems. It is referred to Current Protocols in Molecular Biology, (Ausubel et al, Eds.,) John Wiley & Sons, NY (current edition) for details.

SARS-COV-2 displays some genetic diversity in the S, N, and ORF1a sequences and it may be desirable to use a peptide or a group of peptides that identifies several strains or subtypes. Thus, it may be useful to provide a mixture (a “cocktail”) of two or more peptides herein. In one embodiment such a mixture comprises at least two, preferably three, more preferably four, more preferably five, more preferably six and more preferably seven peptides selected from peptides that comprise or consist of SEQ ID NO 1 to SEQ ID NO 22. In one embodiment the sequences are selected from SEQ ID NO 1 to SEQ ID NO 8, but the present invention makes provision for the inclusion of any of the novel linear epitopes of the invention to be used in combination, e.g. any of the peptides included in Tables 1, 3, or 4, viz. SEQ ID NO 1-377.

One or more peptides may be included in a kit. The kit may be used for diagnosis as described herein. A kit may comprise one or more peptides or mixtures thereof, binding buffer, and detection agents such as a secondary antibody. The kit can include a substrate that immobilises the peptide, such as a solid support, such as microtiter plates, such as ELISA plates to which the peptide(s) of the invention have been pre-adsorbed, various diluents and buffers, labelled conjugates or other agents for the detection of specifically bound antigens or antibodies, such as secondary antibodies, and other signal-generating reagents, such as enzyme substrates, cofactors and chromogens. Other suitable components of a kit can easily be determined by one of skill in the art.

### EXAMPLES

### Materials and Methods

### Patients and Clinical Samples

Patient samples were obtained from the Infectious Diseases Unit, Sahlgrenska University Hospital, between January and June of 2020. Patients were defined as SARS-COV-2 infected by state-of-the-art SARS-COV-2 PCR testing. A serum sample was obtained from patients admitted to the hospital due to COVID-19 symptoms. At the time of admission, the date of symptom onset was noted, and the patient was included in the study cohort if they tested positive by the SARS-COV-2 PCR test. The study was approved by the Human Research Ethics Review Board of Västra Götaland. Pre-pandemic samples were obtained from the same infectious disease unit, and consisted of samples from patients admitted before the onset of the pandemic. In total, 22 SARS-COV-2 infected patients sampled between 14 and 51 days after symptom onset, and 9 pre-pandemic patients were included in the study.

### Mapping of Linear B-Cell Epitopes

Antibody-responses to SARS-COV-2-peptides were assayed using peptide array analysis. Medium-density arrays were created using inkjet-assisted on-chip synthesis technology. On these array chips, 3875 different 12-amino acid (12-mer) SARS-COV-2 peptides were spotted onto each chip. Peptide sequences were from the Wuhan-Hu-1 strain of SARS-COV-2, accession NC_045512.2. The peptide sequences selected were sequential and overlapping and were spanning the entire proteome of SARS-COV-2; for protein S, 11 amino acids overlap between each peptide was used, while 8 aa overlap was used for the remaining proteins. To map antibody-binding to each peptide, each array was incubated with a 1/1000-dilution of a pool of 3 different serum samples from the same disease group, followed by washing and subsequent incubation by Cy3-conjugated rabbit anti-human-IgG and rabbit Cy5-conjugated anti-human-IgG antibodies. Finally, fluorescence image scanning and digital image analysis was performed to detect antibody-binding to each of the peptides on the chip. Chip printing and antibody analysis was performed by way of a commercial service by the company PEPperPRINT (Heidelberg, Germany). The background was detected by preincubating the array with secondary antibodies and measuring binding intensity. Stringent cut-off criteria for identification of linear B-cell epitopes were used by the Applicant, in order to identify epitopes that are useful for diagnostic purposes. These criteria included setting the threshold for binding to a peptide by a serum sample to be 3 SD above the median of the background, using log-transformed data. Furthermore, the criterion to be defined as an epitope was that a sequence stretch had to have at least 3 consecutive peptides above background in at least two different sample pools. If epitopes thus defined had overlapping borders they were finally joined and regarded as one continuous epitope.

Most epitopes were spanning several tested peptides, and the exact location of the bulk of the epitope response varied among different samples. To compare the responses to epitopes between samples the Applicant used the peak value response—for each sample, the Applicant used the peptide binding score that was highest among all the peptides spanning that epitope. The Applicant cross-referenced the sequences of the epitopes they had identified to known SARS-COV-2 epitopes from the Immune Epitope Database (IEDB—www.iedb.org) (1) as at 20 Nov. 2020.

### Results

### Protein S of SARS-COV-2 has 21 Linear B-Cell Epitopes

The Applicant first mapped all linear B-cell epitopes of the SARS-COV-2 protein S by testing pooled sera for binding to S-protein peptides in a peptide array. Using the stringent cut-off criteria defined hereinabove, the Applicant identified 21 linear epitopes of protein S that were used by at least two of the 7 serum sample pools tested. The average length of the epitopes were 17 amino acids. Of these, 90% were IgG epitopes (n=19) 57% were IgA epitopes (n=11), and 48% were both IgG and IgA epitopes (n=10); see Table 1. According to protein S domain boundaries described by Barnes et al (3), the Applicant identified epitopes both in the S1 and S2 domains (Table 1). The S1 domain had 12 epitopes (SEQ ID NO 214-225), located in all subdomains S1^{A-D}, including 4 epitopes in the receptor binding domain (S1^{B}/RBD) (SEQ ID NO 219-222). There were 9 epitopes in the S2 domain (SEQ ID NO 226-234), spanning subdomains S2^{UH}, S2^{FP}, S2^{HR1}, S2^{BH}, S2^{HR2}, and S2^{CT} (Table 1).

TABLE 1
Linear B-cell epitopes of SARS-COV-2 proteins

| | | | | | | | | IgG- | | IgA- | |
|---|---|---|---|---|---|---|---|---|---|---|---|
| SEQ | | Epi- | | Amino acid | | | | antibodies | | antibodies | |
| ID | Prot | tope | Domain | sequence | Start | End | Class | fc¹ | AUC² | fc | AUC |
| 214 | S | S_001 | S1A | FNDGVY | 86 | 91 | IgA | n.a | | 0.6 | |
| 215 | S | S_002 | S1A | CEFQFCNDPFLG | 131 | 142 | IgG | 0.9 | | n.a | |
| 216 | S | S_003 | S1A | KSWMESEFRVYSSAN | 150 | 194 | IgG, | 1.5 | | 1.4 | |
| | | | | NCTFEYVSQPFLMDL | | | IgA | | | | |
| | | | | EGKQGNFKNLREFVF | | | | | | | |
| 217 | S | S_004 | S1A | SALEPLVDLPIGINIT | 221 | 236 | IgG, | 5.6 | | 2 | |
| | | | | | | | IgA | | | | |
| 218 | S | S_005 | S1A | KYNENGTITDAVDCAL | 278 | 298 | IgG, | 6.2 | 0.87 | 7.1 | 0.84 |
| | | | | DPLSE | | | IgA | | | | |
| 219 | S | S_006 | S1B/R | NVYADSF | 394 | 400 | IgG | 2 | 0.59 | n.a | 0.55 |
| | | | BD | | | | | | | | |
| 220 | S | S_007 | S1B/R | PDDFT | 426 | 430 | IgG | 0.5 | 0.80 | n.a | 0.69 |
| | | | BD | | | | | | | | |
| 221 | S | S_008 | S1B/R | FERDI | 464 | 468 | IgG | 1.3 | 0.67 | n.a | 0.51 |
| | | | BD | | | | | | | | |
| 222 | S | S_009 | S1B/R | NGVEGFNCYFP | 481 | 49 | IgG | 2.1 | 0.68 | n.a | 0.73 |
| | | | BD | | | | | | | | |
| 223 | S | S_010 | S1C | GVLTESNKKFLPFQQF | 550 | 586 | IgG, | 3.9 | 0.87 | 2.3 | 0.82 |
| | | | | GRDIADTTDAVRDPQ | | | IgA | | | | |
| | | | | TLEILD | | | | | | | |
| 224 | S | S_011 | S1D | PGTNTSNQVAVLYQD | 600 | 617 | IgG | 2.3 | 0.50 | n.a | 0.53 |
| | | | | VNC | | | | | | | |
| 225 | S | S_012 | S1D | IGAEHVNNSYECDIPI | 651 | 666 | IgG, | 0.7 | | 1 | |
| | | | | | | | IgA | | | | |
| 226 | S | S_013 | S2-UH | ALTGIAVEQDKNTQE | 766 | 780 | IgG, | 3.5 | | 1.4 | |
| | | | | | | | IgA | | | | |
| 227 | S | S_014 | S2 | KTPPIKDFGGFNFSQI | 790 | 808 | IgG | 1.4 | | n.a | |
| | | | | LPD | | | | | | | |
| 228 | S | S_015 | S2 | SKRSFIEDLLFN | 813 | 824 | IgG | 1.9 | 0.73 | n.a | 0.60 |
| 229 | S | S_016 | S2 | QYGDCLGDI | 836 | 844 | IgG | 1.7 | 0.65 | n.a | 0.51 |
| 230 | S | S_017 | S2 | SRLDKVEAEVQID | 982 | 994 | IgG, | 6.6 | | 3.7 | |
| | | | | | | | IgA | | | | |
| 231 | S | S_018 | S2 | FPREGV | 1089 | 1094 | IgA | n.a | | 1.2 | |
| 232 | S | S_019 | S2 | NNTVYDPLQPELDSF | 1134 | 1156 | IgG, | 2.6 | 0.70 | 1.8 | 0.65 |
| | | | | KEELDKYF | | | IgA | | | | |
| 233 | S | S_020 | S2 | PDVDLGDISGINASVV | 1162 | 1208 | IgG, | 2 | 0.52 | 1.4 | 0.66 |
| | | | | NIQKEIDRLNEVAKNL | | | IgA | | | | |
| | | | | NESLIDLQELGKYEQ | | | | | | | |
| 234 | S | S_021 | S2- | CSCGSCCKFDEDDSE | 1248 | 1271 | IgG, | 2.8 | 0.89 | 0.8 | 0.74 |
| | | | CT | PVLKGVKLH | | | IgA | | | | |
| 235 | N | N_001 | | TFGGPSDSTGSNQNG | 16 | 48 | IgG, | 2.5 | 0.86 | 1.4 | 0.75 |
| | | | | ERSGARSKQRRPQGL | | | IgA | | | | |
| | | | | PNN | | | | | | | |
| 236 | N | N_002 | | KFPRGQGVPINTNSS | 65 | 89 | IgG, | 0.3 | | 1.6 | |
| | | | | PDDQIGYYRR | | | IgA | | | | |
| 237 | N | N_003 | | YLGTGPEAGLPYGAN | 112 | 132 | IgG | 1.5 | | n.a | |
| | | | | KDGIIW | | | | | | | |
| 238 | N | N_004 | | ANNAAIVLQLPQGTTL | 152 | 173 | IgG | 1 | | n.a | |
| | | | | PKGFYA | | | | | | | |
| 239 | N | N_005 | | SQASSRSSSRSRNSS | 180 | 197 | IgG, | 0.2 | 0.49 | 0.1 | 0.50 |
| | | | | RNS | | | IgA | | | | |
| 240 | N | N_006 | | LLLLDRLNQLESKROK | 221 | 288 | IgG, | 5.5 | 0.87 | 1.4 | 0.71 |
| | | | | RTATKAYNVTQAFGR | | | IgA | | | | |
| | | | | RGPEQTQGNFGD | | | | | | | |
| 241 | N | N_007 | | TVTKKSAAEASKKPR | 245 | 293 | IgG | 1.7 | 0.88 | n.a | 0.73 |
| | | | | QKRTATKAYNVTQAF | | | | | | | |
| | | | | GRRGPEQTQGNFGD | | | | | | | |
| | | | | QELIR | | | | | | | |
| 242 | N | N_008 | | SAFFGMSRIG | 312 | 321 | IgG | 0.6 | | n.a | |
| 243 | N | N_009 | | AIKLDDKDPN | 336 | 345 | IgG | 0.7 | | n.a | |
| 244 | N | N_010 | | YKTFPPTEPKKDKKKK | 360 | 409 | IgG, | 1.7 | 0.92 | 1.1 | 0.79 |
| | | | | ADETQALPQRQKKQQ | | | IgA | | | | |
| | | | | TVTLLPAADLDDFSKQ | | | | | | | |
| | | | | LQQ | | | | | | | |
| 245 | M | M_001 | | MADSNGTITVEEL | 1 | 13 | IgG, | 6.6 | 0.78 | 4.3 | 0.85 |
| | | | | | | | IgA | | | | |
| 246 | M | M_002 | | ILTRPLLESE | 128 | 137 | IgA | n.a | | 0.9 | |
| 247 | M | M_003 | | LGRCDIKDLPKEITV | 156 | 170 | IgG | 2 | 0.61 | n.a | 0.63 |
| 248 | M | M_004 | | AGDSGFAAYS | 188 | 197 | IgG | 0.5 | | n.a | |
| 249 | E | E_001 | | MYSFVSEETGTLIVNS | 1 | 17 | IgA | n.a | | 1.4 | |
| | | | | V | | | | | | | |
| 250 | OR | ORF1a | NSP1 | VRGFGDSVEEVLSEA | 28 | 65 | IgG, | 2 | | 1.2 | |
| | F1a | b_001 | | RQHLKDGTCGLVEVE | | | IgA | | | | |
| | b | | | KGVLPQLE | | | | | | | |
| 251 | OR | ORF1a | NSP1 | VEKGVLPQLE | 56 | 65 | IgG | 1.4 | 0.48 | n.a | 0.40 |
| | F1a | b_002 | | | | | | | | | |
| | b | | | | | | | | | | |
| 252 | OR | ORF1a | NSP1 | ARTAPHGHVMVELVA | 76 | 105 | IgG, | 1.5 | | 2.5 | |
| | F1a | b_003 | | ELEGIQYGRSGETLG | | | IgA | | | | |
| | b | | | | | | | | | | |
| 253 | OR | ORF1a | NSP1 | SGETLGVLVP | 100 | 109 | IgG | 5 | | n.a | |
| | F1a | b_004 | | | | | | | | | |
| | b | | | | | | | | | | |
| 254 | OR | ORF1a | NSP1 | GGHSYGADLKSFDLG | 132 | 177 | IgG, | 1.1 | 0.72 | 3.4 | 0.59 |
| | F1a | b_005 | | DELGTDPYEDFQENW | | | IgA | | | | |
| | b | | | NTKHSSGVTRELMRE | | | | | | | |
| | | | | L | | | | | | | |
| 255 | OR | ORF1a | NSP2 | RYVDNNFCGPDGYPL | 184 | 201 | IgG, | 1.6 | | 0.7 | |
| | F1a | b_006 | | ECI | | | IgA | | | | |
| | b | | | | | | | | | | |
| 256 | OR | ORF1a | NSP2 | TKRGVYCCREHEHEI | 224 | 257 | IgG, | 0.5 | 0.77 | 0.5 | 0.62 |
| | F1a | b_007 | | AWYTERSEKSYELQT | | | IgA | | | | |
| | b | | | PFEI | | | | | | | |
| 257 | OR | ORF1a | NSP2 | DTFNGECPNF | 264 | 273 | IgG | 0.8 | | n.a | |
| | F1a | b_008 | | | | | | | | | |
| | b | | | | | | | | | | |
| 258 | OR | ORF1a | NSP2 | SEVGPEHSLAEYHNE | 376 | 393 | IgG, | 0.5 | | 0.3 | |
| | F1a | b_009 | | SGL | | | IgA | | | | |
| | b | | | | | | | | | | |
| 259 | OR | ORF1a | NSP2 | TGVVGEGSEGLNDNL | 436 | 453 | IgG, | 1.4 | | 1.5 | |
| | F1a | b_010 | | LEI | | | IgA | | | | |
| | b | | | | | | | | | | |
| 260 | OR | ORF1a | NSP2 | NINIVGDFKLNEEIAIIL | 460 | 477 | IgG | 1.1 | | 1.3 | |
| | F1a | b_011 | | | | | IgA | | | | |
| | b | | | | | | | | | | |
| 261 | OR | ORF1a | NSP2 | VYEKLKPVLDWLEEKF | 620 | 645 | IgG, | 0.7 | | 0.9 | |
| | F1a | b_012 | | KEGVEFLRDG | | | IgA | | | | |
| | b | | | | | | | | | | |
| 262 | OR | ORF1a | NSP2 | HSKGLYRKCVKSRE | 712 | 725 | IgA | n.a | | 2.3 | |
| | F1a | b_013 | | | | | | | | | |
| | b | | | | | | | | | | |
| 263 | OR | ORF1a | NSP2 | PKEIIFLEGETLPTEVL | 736 | 781 | IgG, | 2 | 0.61 | 4.2 | 0.63 |
| | F1a | b_014 | | TEEVVLKTGDLQPLEQ | | | IgA | | | | |
| | b | | | PTSEAVEAPLVGT | | | | | | | |
| 264 | OR | ORF1a | NSP3 | PTKVTFGDDTVIEVQG | 820 | 853 | IgG, | 2.7 | | 0.1 | |
| | F1a | b_015 | | YKSVNITFELDERI | | | IgA | | | | |
| | b | | | | | | | | | | |
| 265 | OR | ORF1a | NSP3 | YTVELGTEVNEFAC | 860 | 873 | IgG, | 1.3 | | 0.9 | |
| | F1a | b_016 | | | | | IgA | | | | |
| | b | | | | | | | | | | |
| 266 | OR | ORF1a | NSP3 | QPVSELLTPLGIDLDE | 884 | 913 | IgG, | 6.6 | | 1.7 | |
| | F1a | b_017 | | WSMATYYLFDESGE | | | IgA | | | | |
| | b | | | | | | | | | | |
| 267 | OR | ORF1a | NSP3 | MYCSFYPPDEDEEEG | 920 | 1001 | IgG, | 0.8 | 0.80 | 1.8 | 0.83 |
| | F1a | b_018 | | DCEEEEFEPSTQYEY | | | IgA | | | | |
| | b | | | GTEDDYQGKPLEFGA | | | | | | | |
| | | | | TSAALQPEEEQEEDW | | | | | | | |
| | | | | LDDDSQQTVGQQDG | | | | | | | |
| | | | | SEDNQTTT | | | | | | | |
| 268 | OR | ORF1a | NSP3 | LEMELTPVVQTIEVNS | 1012 | 1029 | IgG, | 5.2 | | 2 | |
| | F1a | b_019 | | FS | | | IgA | | | | |
| | b | | | | | | | | | | |
| 269 | OR | ORF1a | NSP3 | DNVYIKNADIVEEAKK | 1036 | 1053 | IgG | 1.1 | 0.75 | n.a | 0.81 |
| | F1a | b_020 | | VK | | | | | | | |
| | b | | | | | | | | | | |
| 270 | OR | ORF1a | NSP3 | NNAMQVESDDYIAT | 1080 | 1093 | IgG, | 0.9 | | 1 | |
| | F1a | b_021 | | | | | IgA | | | | |
| | b | | | | | | | | | | |
| 271 | OR | ORF1a | NSP3 | VCVDTVRTNVYLAVF | 1164 | 1205 | IgG, | 1.5 | 0.69 | 1.3 | 0.42 |
| | F1a | b_022 | | DKNLYDKLVSSFLEMK | | | IgA | | | | |
| | b | | | SEKQVEQKIAE | | | | | | | |
| 272 | OR | ORF1a | NSP3 | KEEVKPFITESKPSVE | 1208 | 1261 | IgG, | 2.7 | | 3.7 | |
| | F1a | b_023 | | QRKQDDKKIKACVEE | | | IgA | | | | |
| | b | | | VTTTLEETKFLTENLLL | | | | | | | |
| | | | | YIDING | | | | | | | |
| 273 | OR | ORF1a | NSP3 | NYITTYPGQGLNGYTV | 1324 | 1345 | IgG, | 5.8 | | 4.7 | |
| | F1a | b_024 | | EEAKTV | | | IgA | | | | |
| | b | | | | | | | | | | |
| 274 | OR | ORF1a | NSP3 | KTTVASLINTLNDLNET | 1428 | 1465 | IgG, | 2.9 | | 0.7 | |
| | F1a | b_025 | | LVTMPLGYVTHGLNLE | | | IgA | | | | |
| | b | | | EAARY | | | | | | | |
| 275 | OR | ORF1a | NSP3 | TAYNGYLTSSSKTPEE | 1484 | 1513 | IgG | 0.2 | | 1.4 | |
| | F1a | b_026 | | HFIETISLAGSYKD | | | IgA | | | | |
| | b | | | | | | | | | | |
| 276 | OR | ORF1a | NSP3 | GIEFLK | 1524 | 1529 | IgA | n.a | | 0.4 | |
| | F1a | b_027 | | | | | | | | | |
| | b | | | | | | | | | | |
| 277 | OR | ORF1a | NSP3 | KPHNSHEGKTFYVLP | 1608 | 1637 | IgG, | 0.5 | | 0.7 | |
| | F1a | b_028 | | NDDTLRVEAFEYYHT | | | IgA | | | | |
| | b | | | | | | | | | | |
| 278 | OR | ORF1a | NSP3 | GQQQTTLKGVEAVMY | 1756 | 1777 | IgG, | 1.9 | 0.79 | 4.1 | 0.72 |
| | F1a | b_029 | | MGTLSYE | | | IgA | | | | |
| | b | | | | | | | | | | |
| 279 | OR | ORF1a | NSP3 | LDGVVCTEIDPKLDNY | 1884 | 1917 | IgG | 1.1 | 0.46 | 0.8 | 0.57 |
| | F1a | b_030 | | YKKDNSYFTEQPIDLV | | | IgA | | | | |
| | b | | | PN | | | | | | | |
| 280 | OR | ORF1a | NSP3 | KFADDL | 1936 | 1941 | IgG | 1.3 | | n.a | |
| | F1a | b_031 | | | | | | | | | |
| | b | | | | | | | | | | |
| 281 | OR | ORF1a | NSP3 | KVTFFPDLNGDVVAID | 1956 | 1992 | IgG, | 0.8 | | 2.9 | |
| | F1a | b_032 | | YKHYTPSFKKGAKLLH | | | IgA | | | | |
| | b | | | KPIVW | | | | | | | |
| 282 | OR | ORF1a | NSP3 | SEEVVENPTIQKDVLE | 2048 | 2078 | IgG | 0.7 | 0.72 | 0.6 | 0.71 |
| | F1a | b_033 | | CNVKTTEVVGDIILK | | | IgA | | | | |
| | b | | | | | | | | | | |
| 283 | OR | ORF1a | NSP3 | AFGLVAEWFL | 2320 | 2329 | IgG | 1 | | n.a | |
| | F1a | b_034 | | | | | | | | | |
| | b | | | | | | | | | | |
| 284 | OR | ORF1a | NSP3 | DTFCAGSTFISDEVAR | 2456 | 2472 | IgG | 0.3 | 0.40 | n.a | 0.32 |
| | F1a | b_035 | | D | | | | | | | |
| | b | | | | | | | | | | |
| 285 | OR | ORF1a | NSP3 | TDQSSYIVDS | 2484 | 2493 | IgG | 1.1 | | n.a | |
| | F1a | b_036 | | | | | | | | | |
| | b | | | | | | | | | | |
| 286 | OR | ORF1a | NSP3 | HSLSHFVNLDNLRAN | 2516 | 2532 | IgG | 0.6 | | n.a | |
| | F1a | b_037 | | NT | | | | | | | |
| | b | | | | | | | | | | |
| 287 | OR | ORF1a | NSP3 | PILLLDQALVSDVGDS | 2568 | 2597 | IgG, | 2 | | 1.3 | |
| | F1a | b_038 | | AEVAVKMFDAYVNT | | | IgA | | | | |
| | b | | | | | | | | | | |
| 288 | OR | ORF1a | NSP3 | GFVDSDVETKDVVEC | 2640 | 2669 | IgG, | 2.3 | | 0.8 | |
| | F1a | b_039 | | LKLSHQSDIEVTGDS | | | IgA | | | | |
| | b | | | | | | | | | | |
| 289 | OR | ORF1a | NSP4 | SEIIGYKAID | 2804 | 2813 | IgG | 0.6 | | n.a | |
| | F1a | b_040 | | | | | | | | | |
| | b | | | | | | | | | | |
| 290 | OR | ORF1a | NSP4 | ADFDTWFSQR | 2832 | 2841 | IgA | n.a | | 0.3 | |
| | Fia | b_041 | | | | | | | | | |
| | b | | | | | | | | | | |
| 291 | OR | ORF1a | NSP4 | YTPSKLIEY | 2897 | 2905 | IgG, | 1.8 | | 3.7 | |
| | F1a | b_042 | | | | | IgA | | | | |
| | b | | | | | | | | | | |
| 292 | OR | ORF1a | NSP4 | KPVPYCYDTNVLEGS | 2928 | 2949 | IgG | 1.2 | | n.a | |
| | F1a | b_043 | | VAYESLR | | | | | | | |
| | b | | | | | | | | | | |
| 293 | OR | ORF1a | NSP4 | SVRVVTTFDSEYCRH | 2972 | 2994 | IgG, | 2.2 | 0.60 | 0.9 | 0.67 |
| | F1a | b_044 | | GTCERSEA | | | IgA | | | | |
| | b | | | | | | | | | | |
| 294 | OR | ORF1a | NSP4 | RSLPGVFCGVDAVNL | 3012 | 3033 | IgG | 1.5 | | n.a | |
| | F1a | b_045 | | LTNMFTP | | | | | | | |
| | b | | | | | | | | | | |
| 295 | OR | ORF1a | NSP4 | FMRFRRAFGEYSHV | 3064 | 3077 | IgG | 0.8 | | n.a | |
| | F1a | b_046 | | | | | | | | | |
| | b | | | | | | | | | | |
| 296 | OR | ORF1a | NSP4 | NGVSFSTFEEAALCTF | 3168 | 3185 | IgG, | 2.9 | 0.63 | 1.8 | 0.49 |
| | Fia | b_047 | | LL | | | IgA | | | | |
| | b | | | | | | | | | | |
| 297 | OR | ORF1a | NSP4 | AMDTTSYREA | 3220 | 3229 | IgG | 1.1 | | n.a | |
| | F1a | b_048 | | | | | | | | | |
| | b | | | | | | | | | | |
| 298 | OR | ORF1a | NSP5 | GLWLDDVVYC | 3292 | 3301 | IgG, | 1.7 | 0.65 | 0.8 | 0.73 |
| | F1a | b_049 | | | | | IgA | | | | |
| | b | | | | | | | | | | |
| 299 | OR | ORF1a | NSP5 | DMLNPNYEDLL | 3311 | 3321 | IgG, | 2.2 | 0.73 | 2.4 | 0.57 |
| | F1a | b_050 | | | | | IgA | | | | |
| | b | | | | | | | | | | |
| 300 | OR | ORF1a | NSP5 | SCGSVGFNIDYDCVS | 3407 | 3421 | IgG, | 1.1 | | 0.8 | |
| | F1a | b_051 | | | | | IgA | | | | |
| | b | | | | | | | | | | |
| 301 | OR | ORF1a | NSP5 | TGVHAGTDLEGNFYG | 3432 | 3449 | IgG, | 1.1 | | 0.8 | |
| | F1a | b_052 | | PFV | | | IgA | | | | |
| | b | | | | | | | | | | |
| 302 | OR | ORF1a | NSP5 | AMKYNYEPLTQDHVDI | 3497 | 3513 | IgG, | 1.3 | | 1.9 | |
| | F1a | b_053 | | L | | | IgA | | | | |
| | b | | | | | | | | | | |
| 303 | OR | ORF1a | NSP5 | ILGSALLEDEFTPFD | 3544 | 3561 | IgG, | 0.9 | | 1.4 | |
| | Fia | b_054 | | | | | IgA | | | | |
| | b | | | | | | | | | | |
| 304 | OR | ORF1a | NSP6 | QSTQWSLFFFLYENA | 3596 | 3613 | IgG, | 1 | | 0.9 | |
| | F1a | b_055 | | FLP | | | IgA | | | | |
| | b | | | | | | | | | | |
| 305 | OR | ORF1a | | LGVYDYLVST | 3808 | 3817 | IgG, | 0.7 | | 1 | |
| | F1a | b_056 | | | | | IgA | | | | |
| | b | | | | | | | | | | |
| 306 | OR | ORF1a | NSP7/ | AVDINKLCEEMLDNRA | 3924 | 3949 | IgG, | 4.3 | | 0.9 | |
| | F1a | b_057 | NSP8 | TLQAIASEFS | | | IgA | | | | |
| | b | | | | | | | | | | |
| 307 | OR | ORF1a | NSP8 | AQEAYEQAVA | 3960 | 3972 | IgG, | 0.9 | | 1.3 | |
| | F1a | b_058 | | | | | IgA | | | | |
| | b | | | | | | | | | | |
| 308 | OR | ORF1a | NSP8 | VLKKLKKSLNVAKS | 3976 | 3989 | IgG, | 4.7 | | 2.8 | |
| | F1a | b_059 | | | | | IgA | | | | |
| | b | | | | | | | | | | |
| 309 | OR | ORF1a | NSP8 | ASALWEIQQVVDAD | 4092 | 4105 | IgG | 0.8 | | n.a | |
| | F1a | b_060 | | | | | | | | | |
| | b | | | | | | | | | | |
| 310 | OR | ORF1a | NSP9 | CTDDNALAYYN | 4163 | 4176 | IgG, | 0.8 | | 1 | |
| | F1a | b_061 | | | | | IgA | | | | |
| | b | | | | | | | | | | |
| 311 | OR | ORF1a | NSP9 | TIYTELEPPCRFVT | 4204 | 4217 | IgG | 2.6 | | n.a | |
| | F1a | b_062 | | | | | | | | | |
| | b | | | | | | | | | | |
| 312 | OR | ORF1a | NSP10 | AITVTPEANMDQES | 4307 | 4320 | IgG | 0.9 | | n.a | |
| | F1a | b_063 | | | | | | | | | |
| | b | | | | | | | | | | |
| 313 | OR | ORF1a | NSP10 | YGCSCDQLREPMLQS | 4379 | 4397 | IgG, | 1.7 | | 1 | |
| | F1a | b_064 | /NSP1 | ADAQ | | | IgA | | | | |
| | b | | 1 | | | | | | | | |
| 314 | OR | ORF1a | NSP11 | TDVVYRAFDIYNDK | 4420 | 4433 | IgG, | 0.9 | | 0.5 | |
| | F1a | b_065 | | | | | IgA | | | | |
| | b | | | | | | | | | | |
| 315 | OR | ORF1a | NSP11 | CCRFQEKDEDDNLID | 4445 | 4482 | IgG, | 1.4 | | 0.5 | |
| | F1a | b_066 | | SYFVVKRHTFSNYQH | | | IgA | | | | |
| | b | | | EETIYNLL | | | | | | | |
| 316 | OR | ORF1a | NSP11 | TFSNYQHEETIYNL | 4468 | 4481 | IgA | n.a | | 1.1 | |
| | F1a | b_067 | | | | | | | | | |
| | b | | | | | | | | | | |
| 317 | OR | ORF1a | NSP11 | VYALRHFDEGNCDTL | 4520 | 4566 | IgG, | 1 | 0.86 | 0.4 | 0.79 |
| | F1a | b_068 | | KEILVTYNCCDDDYFN | | | IgA | | | | |
| | b | | | KKDWYDFVENPDILR | | | | | | | |
| 318 | OR | ORF1a | NSP11 | GIVGVLTLDNQDLNGN | 4592 | 4617 | IgG, | 1.3 | 0.72 | 2.8 | 0.71 |
| | F1a | b_069 | | WYDFGDFIQT | | | IgA | | | | |
| | b | | | | | | | | | | |
| 319 | OR | ORF1a | NSP11 | KYDFTEERLKLFDRYF | 4664 | 4681 | IgG, | 2 | | 1.1 | |
| | F1a | b_070 | | KY | | | IgA | | | | |
| | b | | | | | | | | | | |
| 320 | OR | ORF1a | NSP11 | QTYHPNCVNCLDDR | 4684 | 4697 | IgG | 1.5 | | n.a | |
| | F1a | b_071 | | | | | | | | | |
| | b | | | | | | | | | | |
| 321 | OR | ORF1a | NSP11 | FRELGVVHNQDVNLH | 4740 | 4757 | IgG | 2.1 | | n.a | |
| | F1a | b_072 | | SSR | | | | | | | |
| | b | | | | | | | | | | |
| 322 | OR | ORF1a | NSP11 | NKDFYDFAVSKGFFK | 4808 | 4829 | IgG, | 4.2 | | 1.3 | |
| | F1a | b_073 | | EGSSVEL | | | IgA | | | | |
| | b | | | | | | | | | | |
| 323 | OR | ORF1a | NSP11 | FFFAQDGNAAISDYDY | 4832 | 4849 | IgG, | 0.9 | | 0.7 | |
| | F1a | b_074 | | YR | | | IgA | | | | |
| | b | | | | | | | | | | |
| 324 | OR | ORF1a | NSP11 | QLLFVVEVVDKYFDCY | 4860 | 4884 | IgG, | 0.9 | 0.55 | 0.6 | 0.54 |
| | F1a | b_075 | | DGGCINANQ | | | IgA | | | | |
| | b | | | | | | | | | | |
| 325 | OR | ORF1a | NSP11 | YYDSMSYEDQDALF | 4907 | 4920 | IgG, | 0.8 | | 0.4 | |
| | F1a | b_076 | | | | | IgA | | | | |
| | b | | | | | | | | | | |
| 326 | OR | ORF1a | NSP11 | AATRGATVVI | 4972 | 4981 | IgG | 0.3 | | n.a | |
| | F1a | b_077 | | | | | | | | | |
| | b | | | | | | | | | | |
| 327 | OR | ORF1a | NSP11 | PHLMGWDYPK | 5004 | 5013 | IgG | 1 | | n.a | |
| | F1a | b_078 | | | | | | | | | |
| | b | | | | | | | | | | |
| 328 | OR | ORF1a | NSP11 | YECLYRNRDVDTDFV | 5120 | 5137 | IgG, | 1.7 | | 1.1 | |
| | F1a | b_079 | | NEF | | | IgA | | | | |
| | b | | | | | | | | | | |
| 329 | OR | ORF1a | NSP11 | MILSDD | 5148 | 5153 | IgG | 2.7 | | n.a | |
| | F1a | b_080 | | | | | | | | | |
| | b | | | | | | | | | | |
| 330 | OR | ORF1a | NSP11 | VKQGDDYVYL | 5212 | 5221 | IgG, | 1.1 | | 0.2 | |
| | F1a | b_081 | | | | | IgA | | | | |
| | b | | | | | | | | | | |
| 331 | OR | ORF1a | NSP11 | GAGCFVDDIVKTDGTL | 5231 | 5253 | IgG, | 1.4 | | 0.6 | |
| | F1a | b_082 | | MIERFVS | | | IgA | | | | |
| | b | | | | | | | | | | |
| 332 | OR | ORF1a | NSP11 | LTKHPNQEYADVFH | 5261 | 5274 | IgG, | 1.5 | | 1.9 | |
| | F1a | b_083 | | | | | IgA | | | | |
| | b | | | | | | | | | | |
| 333 | OR | ORF1a | NSP11 | TNDNTSRYWEPEFYE | 5300 | 5317 | IgG, | 1 | 0.60 | 0.8 | 0.58 |
| | F1a | b_084 | | AMY | | | IgA | | | | |
| | b | | | | | | | | | | |
| 334 | OR | ORF1a | NSP12 | RRPFLCCKC | 5345 | 5353 | IgG | 0.4 | | n.a | |
| | F1a | b_085 | | | | | | | | | |
| | b | | | | | | | | | | |
| 335 | OR | ORF1a | NSP12 | SDNVTDFNAIATCDW | 5424 | 5460 | IgG | 2.4 | | n.a | |
| | F1a | b_086 | | TNAGDYILANTCTERL | | | | | | | |
| | b | | | KLFAAE | | | | | | | |
| 336 | OR | ORF1a | NSP12 | TEETFKLSYGIATVRE | 5465 | 5493 | IgG, | 1.5 | | 1.7 | |
| | F1a | b_087 | | VLSDRELHLSWEV | | | IgA | | | | |
| | b | | | | | | | | | | |
| 337 | OR | ORF1a | NSP12 | IGEYTFEKGDY | 5519 | 5532 | IgG, | 0.4 | | 0.4 | |
| | F1a | b_088 | | | | | IgA | | | | |
| | b | | | | | | | | | | |
| 338 | OR | ORF1a | NSP12 | QEHYVRITGLYPTLNIS | 5567 | 5586 | IgG | 0.3 | | n.a | |
| | F1a | b_089 | | DEF | | | | | | | |
| | b | | | | | | | | | | |
| 339 | OR | ORF1a | NSP12 | FCTVNALPETTADIVV | 5681 | 5706 | IgG, | 2.4 | 0.78 | 1.6 | 0.83 |
| | F1a | b_090 | | FDEIS | | | IgA | | | | |
| | b | | | | | | | | | | |
| 340 | OR | ORF1a | NSP12 | PRTLLTKGTLEPEYFN | 5732 | 5748 | IgA | n.a | | 5.6 | |
| | F1a | b_091 | | S | | | | | | | |
| | b | | | | | | | | | | |
| 341 | OR | ORF1a | NSP12 | TCRRCPAEIVDTVSAL | 5764 | 5781 | IgG | 5.2 | 0.59 | n.a | 0.63 |
| | F1a | b_092 | | VY | | | | | | | |
| | b | | | | | | | | | | |
| 342 | OR | ORF1a | NSP12 | QIGVVREFLT | 5816 | 5825 | IgA | n.a | | 3.1 | |
| | F1a | b_093 | | | | | | | | | |
| | b | | | | | | | | | | |
| 343 | OR | ORF1a | NSP12 | TVDSSQGSEYDYVI | 5856 | 5873 | IgG, | 1.5 | 0.72 | 1.1 | 0.65 |
| | F1a | b_094 | | | | | IgA | | | | |
| | b | | | | | | | | | | |
| 344 | OR | ORF1a | NSP12 | MSDRDLYDKLOFTS | 5900 | 5913 | IgG | 0.2 | | n.a | |
| | F1a | b_095 | | | | | | | | | |
| | b | | | | | | | | | | |
| 345 | OR | ORF1a | NSP13 | NYQVNGYPNMFITRE | 5988 | 6025 | IgG, | 5.6 | | 1.2 | |
| | F1a | b_096 | | EAIRHVRAWIGFDVEG | | | IgA | | | | |
| | b | | | CHATREA | | | | | | | |
| 346 | OR | ORF1a | NSP13 | TGYVDTPNNTDFSRV | 6047 | 6061 | IgG | 1.9 | | n.a | |
| | F1a | b_097 | | | | | | | | | |
| | b | | | | | | | | | | |
| 347 | OR | ORF1a | NSP13 | WHHSIGFDYVYNPFMI | 6152 | 6169 | IgG, | 1.4 | | 0.9 | |
| | F1a | b_098 | | DV | | | IgA | | | | |
| | b | | | | | | | | | | |
| 348 | OR | ORF1a | NSP13 | DWTIEYPIIGDELK | 6216 | 6229 | IgG, | 1.4 | 0.81 | 0.5 | 0.75 |
| | F1a | b_099 | | | | | IgA | | | | |
| | b | | | | | | | | | | |
| 349 | OR | ORF1a | NSP13 | ADKFPV | 6248 | 6253 | IgG | 0.7 | | n.a | |
| | F1a | b_100 | | | | | | | | | |
| | b | | | | | | | | | | |
| 350 | OR | ORF1a | NSP13 | QADVEWKFY | 6268 | 6276 | IgG, | 2 | | 0.8 | |
| | Fia | b_101 | | | | | IgA | | | | |
| | b | | | | | | | | | | |
| 351 | OR | ORF1a | NSP13 | KAYKIEELFYSYATHS | 6284 | 6318 | IgG, | 0.8 | | 0.8 | |
| | F1a | b_102 | | DKFTDGVCLFWNCNV | | | IgA | | | | |
| | b | | | DRYP | | | | | | | |
| 352 | OR | ORF1a | NSP13 | VCRHHANEYR | 6408 | 6417 | IgG, | 0.9 | | 0.3 | |
| | F1a | b_103 | | | | | IgA | | | | |
| | b | | | | | | | | | | |
| 353 | OR | ORF1a | NSP14 | KGHFDGQQGEVPVSII | 6464 | 6481 | IgG, | 1.2 | | 1.6 | |
| | F1a | b_104 | | NN | | | IgA | | | | |
| | b | | | | | | | | | | |
| 354 | OR | ORF1a | NSP14 | YTKVDGVDVELFENK | 6484 | 6504 | IgG | 6.3 | | n.a | |
| | F1a | b_105 | | TTLPVN | | | | | | | |
| | b | | | | | | | | | | |
| 355 | OR | ORF1a | NSP14 | IKPVPEVKILNNLGVDI | 6515 | 6541 | IgG, | 4.2 | 0.42 | 0.2 | 0.22 |
| | F1a | b_106 | | AANTVIWDYK | | | IgA | | | | |
| | b | | | | | | | | | | |
| 356 | OR | ORF1a | NSP14 | ANTVIWDYK | 6533 | 6541 | IgG | 1.1 | | n.a | |
| | F1a | b_107 | | | | | | | | | |
| | b | | | | | | | | | | |
| 357 | OR | ORF1a | NSP14 | TETICAPLTVFFDGRV | 6564 | 6585 | IgG, | 1.8 | | 0.5 | |
| | F1a | b_108 | | DGQVDL | | | IgA | | | | |
| | b | | | | | | | | | | |
| 358 | OR | ORF1a | NSP14 | KPRSQMEIDFLELAMD | 6656 | 6693 | IgG, | 1.7 | 0.70 | 0.8 | 0.77 |
| | F1a | b_109 | | EFIERYKLEGYAFEHIV | | | IgA | | | | |
| | b | | | YGDFS | | | | | | | |
| 359 | OR | ORF1a | NSP14 | IGLAKRFKESPFELED | 6704 | 6725 | IgG, | 1.2 | 0.69 | 0.7 | 0.69 |
| | F1a | b_110 | | FIPMDS | | | IgA | | | | |
| | b | | | | | | | | | | |
| 360 | OR | ORF1a | NSP14 | VCSVIDLLLDDFVEI | 6743 | 6757 | IgG, | 7.7 | 0.72 | 1.9 | 0.71 |
| | F1a | b_111 | | | | | IgA | | | | |
| | b | | | | | | | | | | |
| 361 | OR | ORF1a | NSP15 | DLQNYGDSAT | 6824 | 6833 | IgG | 1.8 | | n.a | |
| | F1a | b_112 | | | | | | | | | |
| | b | | | | | | | | | | |
| 362 | OR | ORF1a | NSP15 | LLVDSDLNDFVSDADS | 6892 | 6929 | IgG, | 1.3 | | 1.4 | |
| | F1a | b_113 | | TLIGDCATVHTANKW | | | IgA | | | | |
| | b | | | DLIISDM | | | | | | | |
| 363 | OR | ORF1a | NSP15 | WTAFVTNVNASSSEA | 6988 | 7029 | IgG | 1.5 | | 1.3 | |
| | F1a | b_114 | | FLIGCNYLGKPREQID | | | IgA | | | | |
| | b | | | GYVMHANYIFW | | | | | | | |
| 364 | OR | ORF1a | NSP15 | NYLGKPREQIDGYV | 7008 | 7021 | IgG | 0.9 | | n.a | |
| | F1a | b_115 | | | | | | | | | |
| | b | | | | | | | | | | |
| 365 | OR | ORF1a | NSP2 | ACHNSEVGPEHSLAE | 372 | 393 | IgG | 0.5 | | n.a | |
| | F1a | _001 | | YHNESGL | | | | | | | |
| 366 | OR | ORF1a | NSP3 | QRKQDDKKIKACVEE | 1224 | 1257 | IgA | n.a | | 1.8 | |
| | F1a | _002 | | VTTTLEETKFLTENLLL | | | | | | | |
| | | | | YI | | | | | | | |
| 367 | OR | ORF1a | NSP10 | YGCSCDQLREPMLQS | 4379 | 4393 | IgG | 1.7 | | n.a | |
| | F1a | _003 | | | | | | | | | |
| 368 | OR | ORF3a | | LYDANYFLCWHTNCY | 140 | 161 | IgG, | 0.8 | | 1.2 | |
| | F3a | _001 | | DYC | | | IgA | | | | |
| 369 | OR | ORF3a | | GDGTTSPISEHDYQ | 172 | 193 | IgG, | 0.7 | | 0.2 | |
| | F3a | _002 | | | | | IgA | | | | |
| 370 | OR | ORF3a | | GVEHVTFFIYNKIVDE | 224 | 275 | IgG | 0.6 | 0.67 | 1.1 | 0.68 |
| | F3a | _003 | | PEEHVQIHTIDGSSGV | | | IgA | | | | |
| | | | | VNPVMEPIYDEPTTTT | | | | | | | |
| | | | | SVPL | | | | | | | |
| 371 | OR | ORF7a | | ALITLATCELYHYQ | 8 | 21 | IgG, | 0.4 | | 0.5 | |
| | F7a | _001 | | | | | IgA | | | | |
| 372 | OR | ORF7a | | SSGTYEGNSPFHPL | 36 | 49 | IgG | 1.8 | | n.a | |
| | F7a | _002 | | | | | | | | | |
| 373 | OR | ORF7a | | PKLFIRQEEVQELYSPI | 84 | 10 | IgG, | 1.3 | | 1 | |
| | F7a | _003 | | F | | | IgA | | | | |
| 374 | OR | ORF7b | | MIELSLIDFYLCF | 1 | 13 | IgG | 1.4 | 0.5 | n.a | 0.63 |
| | F7b | _001 | | | | | | | | | |
| 375 | OR | ORF7b | | LELQDHNETCHA | 32 | 43 | IgA | n.a | | 0.8 | |
| | F7b | _002 | | | | | | | | | |
| 376 | OR | ORF8 | | HQPYVVDDPC | 28 | 37 | IgG, | 1 | | 0.8 | |
| | F8 | _001 | | | | | IgA | | | | |
| 377 | OR | ORF10 | | VVNFNLT | 32 | 38 | IgG | 1.2 | | n.a | |
| | F10 | _001 | | | | | | | | | |

¹fc: ratio for the response of SARS-CoV-2-infected vs non-infected sample pools. Samples were pooled (n = 3 samples per pool), and the median of 7 pools of samples from infected individuals were compared to one pool of samples from uninfected (pre-pandemic) individuals.
²AUC: Diagnostic accuracy (Receiver Operating Characteristic Area Under the Curve) for response of SARS-CoV-2-infected (n = 22) vs non-infected (n = 12) samples; samples from infected individuals were obtained 14 days or more after symptom onset. Samples were tested individually, in an experiment separate from the screening experiment that defined the epitopes. Epitopes that were not tested using individual samples lack values.
³n.a: Not applicable. The ratio of response was calculated only for the relevant antibody class(es).

As verification of the veracity of the Applicant's invention, a comparison was made between S epitopes previously identified and those identified using the techniques of the present invention by the Applicant. The Applicant's designed peptides and peptide fragments identified 54% of all S protein epitopes that had been reported in the IEDB database (as of Nov. 12, 2020). Of note, two of the identified epitopes (SEQ ID NO 223 and SEQ ID NO 228) had previously been confirmed as containing neutralising epitopes (4).

Surprisingly, the majority of linear B-cell epitopes for SARS-COV-2 were found by the Applicant to be located in proteins other than protein S, using the methodology of the present invention. Protein S has the ability to bind to and infect host cells, and therefore most research groups have focused their efforts on the immune responses to protein S. However, following analysis of the results presented herein, the Applicant is of the opinion that the responses to other antigens are of likely importance for pathogenicity and could also provide significant diagnostic capabilities for SARS-COV-2 infection and prediction of disease progression. The Applicant designed peptides and peptide fragments to map the linear B-cell epitopes of the other nine SARS-COV-2 proteins, using a sequence overlap of 8 amino acids for peptides of 12 amino acid length. The Applicant identified 143 linear B-cell epitopes in these proteins (SEQ ID NO 235-377), with an average length of 21 amino acids (Table 1). These epitopes were relatively evenly distributed throughout the SARS-COV-2 proteome, with one epitope per around 60 amino acids overall.

The ORF1ab polyprotein is the largest entity in the genome, and here the Applicant identified 115 epitopes (SEQ ID NOS 250-364), in accordance with the invention.

In addition, there were ten epitopes in the nucleocapsid protein (SEQ ID NOS 235-244), four in the membrane glycoprotein (SEQ ID NOS 245-248), three in each of the ORF1a (SEQ ID NOS 365-367), ORF3a (SEQ ID NOS 368-370) and ORF7a (SEQ ID NOS 371-373) proteins, two in the ORF7b (SEQ ID NOS 374-375) and one each in ORF8 (SEQ ID NOS 376), ORF10 (SEQ ID NO 377) and the envelope protein (SEQ ID NOS 249).

Out of the 143 non-spike epitopes identified, 93% (n=133) were IgG epitopes and 69% (n=98) were IgA epitopes; 62% of the epitopes (n=88) were used by both IgG and IgA, in accordance with one aspect of the invention.

Importantly, some of the amino acid mutations of the recently emerging B.1.1.7 strain of SARS-COV-2 are located in epitopes the Applicant has identified in accordance with the invention. For example, A570D and S982A of protein S are located in epitopes S_010 (SEQ ID NOS 7, 18, 76, 77 and 223) and S_017 (SEQ ID NO 230), respectively, T10011 of ORF1ab and S235F of protein N are located in epitopes ORF1ab_018 (SEQ ID NO 267) and N_006 (SEQ ID NOs 139 and 240). The Applicant has found that by varying the amino acid sequences of these epitopes in accordance with the invention, diagnostics are produced that can distinguish between infections of these strains.

Furthermore, the E484K mutation of the emerging strain B.1.351 is located in epitope S_009 (SEQ ID NOS 63, 64 and 222) of protein S, indicating that infection with this strain can also be distinguished by varying peptide sequences according to the methodologies in accordance with the invention.

Sera from Individuals Never Exposed to SARS-COV-2 have Antibodies to a Large Fraction of SARS-COV-2 Epitopes

To identify areas of the SARS-COV-2 proteome that could be used for accurate assessment of antibody-responses in infected vs uninfected individuals, and thereby identify current or past SARS-COV-2 infection, the Applicant tested a group of serum samples taken before the pandemic (pre-COVID-19 samples). In as much as 32% of the SARS-COV-2 peptides (n=1249 out of 3875 peptides) there was a response above the background cut-off in either IgG or IgA in these pre-COVID-19 samples.

The relative differences between infected and pre-COVID-19 samples for each identified epitope are indicated in Table 1. Within the identified epitopes, the pre-COVID-19 samples had an IgG response higher than the median infected sample in 34% of the epitopes (n=55) and an IgA response higher than the median infected sample in 45% (n=74) of the epitopes (Table 2).

In the antigens of main relevance for currently available commercial COVID-19 antibody tests—protein S and the nucleocapsid protein—14-40% of the epitopes had a higher response in pre-COVID samples than in the median infected samples (Table 2). This highlights that unless serological tests are based on a precision-immunology approach whereby only carefully selected epitopes are used, current tests of which the Applicant is aware run a high risk of creating inaccurate outcomes containing high false-positive rates.

The Applicant's data presented herein shows that the small, highly discriminatory selection of peptides of the present invention have the potential to create a high accuracy test even if only a small, well-defined subset of B-cell epitopes are used—those epitopes for which there is a response only in SARS-COV-2-infected patients and not in pre-pandemic samples (see Table 2 and columns fc (IgG) and fc (IgA) in Table 1). These epitopes, that constitute only 66% of all IgG epitopes and 55% of all IgA epitopes, can be used either alone or in combination to create accurate serology diagnostics methods.

TABLE 2
Epitopes with pre-existing antibodies.

| | | Epitopes with pre- | |
|---|---|---|---|
| | Total | existing response¹ | |
| Protein | epitopes | IgG | IgA |

| | | | | | |
|---|---|---|---|---|---|
| Protein S | 21 | 3 | (14%) | 6 | (29%) |
| Protein N | 10 | 4 | (40%) | 4 | (40%) |
| Protein M | 4 | 2 | (50%) | 3 | (75%) |
| ORF1ab | 115 | 39 | (34%) | 53 | (46%) |
| Other proteins | 14 | 7 | (50%) | 8 | (57%) |
| All proteins | 164 | 55 | (34%) | 74 | (45%) |

¹Epitopes with pre-existing responses were defined as those epitopes where a pool of samples from before the pandemic had a response higher than the median response of pools (n = 7) of samples from SARS-CoV-2-infected individuals.

Four Epitope(s) from Protein S, Two Epitopes from Protein N and Three Epitopes from ORF1a are Useful for Diagnosis when Analysed Individually

To identify the peptides that are most useful for diagnosis of infection, the Applicant analysed individual patient sera in new peptide arrays. These arrays contained peptides covering the most strongly reactive epitopes from the screening phase, in addition to a number of peptides from the Receptor Binding Domain (RBD) of protein S (n=213 peptides in total). The Applicant tested the ability of all these peptides to diagnose SARS-CoV-2 infection by testing IgG and IgA antibody-binding to each peptide for samples from SARS-COV-2 infected individuals obtained at 14 days or more after onset of symptoms (n=22) and from samples obtained before the pandemic (n=12). Most of these samples were the same as tested in the first set of arrays, but now these samples were tested individually instead of in a pooled fashion, in order to estimate the frequency of use of each epitope. The Applicant determined the diagnostic accuracy by calculating the Receiver Operating Characteristic Area Under the Curve (AUC) for each of these peptides when comparing SARS-COV-2-infected with pre-pandemic samples. Among the tested peptides, the Applicant found an AUC of at least 0.90 for 5 peptides (SEQ ID NOS 1-5), and an AUC of at least 0.80 for 19 peptides (SEQ ID NOS 1-19), when measuring IgG antibody levels (Table 3). For accuracy levels of all tested peptides see Table 4. The highly discriminatory peptides of the invention belonged to protein S (eight peptides within epitopes S_005, S_010, S_019 and S_021; viz. SEQ ID NOS 2, 4, 6, 7, 11, 13, 15 and 18), protein N (five peptides within epitopes N_006 and N_010; viz. SEQ ID NOS 1, 3, 5, 12 and 19) and the ORF1ab polyprotein (six peptides within epitopes ORF1a_005, ORF1a_018 and ORF1a_068; viz. SEQ ID NOS 8, 9, 10, 14, 16 and 17).

For IgA responses, there were ten peptides with an AUC of at least 0.80 but none with an AUC of 0.90 or above (Table 3). The IgA-discriminatory peptides belonged to S_005, S_010, S_021, N_010, ORF1a_018, 068 and ORF1a_090 (SEQ ID NOS 1, 2, 3, 10, 13, 15, 16, 20, 21 and 22, respectively).

TABLE 3
The most discriminatory peptides of the invention
for diagnosing SARS-CoV-2 infection

| SEQ | Pro- | Domain/ | | Amino acid | Posi- | AUC | AUC |
|---|---|---|---|---|---|---|---|
| ID | tein | protein | Epitope | sequence | tion¹ | IgG² | IgA³ |
| 1 | N | | N_010 | TEPKKDKKKKAD | 365 | 0.94 | 0.81 |
| 2 | S | S1C | S_010 | VRDPQTLEILDI | 575 | 0.94 | 0.80 |
| 3 | N | | N_010 | FPPTEPKKDKKK | 362 | 0.92 | 0.81 |
| 4 | S | S1C | S_010 | TDAVRDPQTLEI | 572 | 0.90 | 0.78 |
| 5 | N | | N_006 | AAEASKKPRQKR | 250 | 0.90 | 0.71 |
| 6 | S | S2-CT | S_021 | CCKFDEDDSEPV | 1252 | 0.88 | 0.71 |
| 7 | S | S1C | S_010 | PFQQFGRDIADT | 560 | 0.88 | 0.58 |
| 8 | ORF1ab | NSP3 | ORF1ab_018 | DDDSQQTVGQQD | 980 | 0.86 | 0.73 |
| 9 | ORF1ab | NSP3 | ORF1ab_018 | LQPEEEQEEDWL | 968 | 0.86 | 0.71 |
| 10 | ORF1ab | NSP11 | ORF1ab_068 | DDDYFNKKDWYD | 4544 | 0.85 | 0.81 |
| 11 | S | S2 | S_019 | QPELDSFKEELD | 1141 | 0.84 | 0.77 |
| 12 | N | | N_006 | AEASKKPRQKRT | 251 | 0.84 | 0.71 |
| 13 | S | S2-CT | S_021 | FDEDDSEPVLKG | 1255 | 0.83 | 0.87 |
| 14 | ORF1ab | NSP3 | ORF1ab_018 | TEDDYQGKPLEF | 950 | 0.83 | 0.65 |
| 15 | S | S1A | S_005 | YNENGTITDAVD | 278 | 0.81 | 0.87 |
| 16 | ORF1ab | NSP3 | ORF1ab_018 | EGDCEEEEFEPS | 932 | 0.81 | 0.80 |
| 17 | ORF1ab | NSP1 | ORF1ab_005 | LGDELGTDPYED | 144 | 0.81 | 0.75 |
| 18 | S | S1C | S_010 | QFGRDIADTTDA | 563 | 0.80 | 0.73 |
| 19 | N | | N_006 | QQQQGQTVTKKS | 238 | 0.80 | 0.54 |
| 20 | ORF1ab | NSP3 | ORF1ab_018 | PDEDEEEGDCEE | 926 | 0.79 | 0.83 |
| 21 | ORF1ab | NSP12 | ORF1ab_090 | ETTADIVVFDEI | 5688 | 0.75 | 0.81 |
| 22 | S | S1C | S_010 | ADTTDAVRDPQT | 569 | 0.73 | 0.80 |

¹Position: The amino acid position of the first amino acid of each peptide within the protein from where it originates.
^{2,3} AUC: Diagnostic accuracy (Receiver Operating Characteristic Area Under the Curve) for antibody-levels to each peptide, comparing samples from SARS-CoV-2-infected (n = 22) vs non-infected (n = 9) individuals. Samples were tested individually, in peptide arrays containing 213 different SARS-CoV-2 peptides. Only peptides with an AUC of 0.80 or above are shown.

Although these individual peptides can accurately identify SARS-COV-2 infection, a combination of several peptides yield an even more robust diagnosis of infection. The Applicant analysed all possible 3-peptide combinations of these discriminatory peptides. This was done by addition of the array scores for each peptide contained in each combination for each patient sample. The accuracies of the 3-peptide combinations were indeed higher than for individual peptides. For IgG, the median AUC of these combinations reached 0.93 (range 0.81-1.00, n=680 combinations) and for IgA, the median AUC reached 0.88 (range 0.84-0.93, n=35 combinations). For 2-peptide combinations, the AUC range was 0.81-0.99 for IgG (n=136 combinations) and 0.79-0.96 (n=21 combinations) for IgA. Taken together, this shows that any of the discriminatory peptides of Table 3 can be used in any 2- or 3-combination to reach high accuracy of infection diagnosis.

TABLE 4
Diagnostic accuracy of all tested SARS-CoV-2 peptides

| SEQ | Pro- | | | Amino acid | Posi- | AUC | AUC |
|---|---|---|---|---|---|---|---|
| ID | tein | Domain | Epitope | sequence | tion | IgG | IgA |
| 23 | S | S1A | | QLPPAYTNSFTR | 22 | 0.48 | 0.37 |
| 24 | S | S1A | | PAYTNSFTRGVY | 25 | 0.35 | 0.61 |
| 25 | S | S1A | | TNSFTRGVYYPD | 28 | 0.52 | 0.50 |
| 26 | S | S1A | | FTRGVYYPDKVF | 31 | 0.54 | 0.57 |
| 27 | S | S1A | | GVYYPDKVFRSS | 34 | 0.61 | 0.51 |
| 28 | S | S1A | | YPDKVFRSSVLH | 37 | 0.45 | 0.48 |
| 29 | S | S1A | S_005 | RTFLLKYNENGT | 272 | 0.63 | 0.49 |
| 30 | S | S1A | S_005 | LLKYNENGTITD | 275 | 0.70 | 0.64 |
| 15 | S | S1A | S_005 | YNENGTITDAVD | 278 | 0.81 | 0.87 |
| 31 | S | S1A | S_005 | NGTITDAVDCAL | 281 | 0.65 | 0.55 |
| 32 | S | S1A | S_005 | ITDAVDCALDPL | 284 | 0.68 | 0.59 |
| 33 | S | S1A | S_005 | AVDCALDPLSET | 287 | 0.75 | 0.75 |
| 34 | S | S1A | S_005 | DCALDPLSETKC | 289 | 0.55 | 0.37 |
| 35 | S | S1A | S_005 | CALDPLSETKCT | 290 | 0.18 | 0.41 |
| 36 | S | S1 | | SETKCTLKSFTV | 296 | 0.31 | 0.36 |
| 37 | S | S1 | | KCTLKSFTVEKG | 299 | 0.48 | 0.40 |
| 38 | S | S1 | | RFPNITNLCPFG | 327 | 0.53 | 0.42 |
| 39 | S | S1B/RB | | NLCPFGEVFNAT | 333 | 0.49 | 0.49 |
| | | D | | | | | |
| 40 | S | S1B/RB | | EVFNATRFASVY | 339 | 0.53 | 0.62 |
| | | D | | | | | |
| 41 | S | S1B/RB | | RFASVYAWNRKR | 345 | 0.57 | 0.59 |
| | | D | | | | | |
| 42 | S | S1B/RB | | AWNRKRISNCVA | 351 | 0.41 | 0.36 |
| | | D | | | | | |
| 43 | S | S1B/RB | | ISNCVADYSVLY | 357 | 0.50 | 0.52 |
| | | D | | | | | |
| 44 | S | S1B/RB | | DYSVLYNSASFS | 363 | 0.55 | 0.48 |
| | | D | | | | | |
| 45 | S | S1B/RB | | NSASFSTFKCYG | 369 | 0.23 | 0.38 |
| | | D | | | | | |
| 46 | S | S1B/RB | | TFKCYGVSPTKL | 375 | 0.36 | 0.32 |
| | | D | | | | | |
| 47 | S | S1B/RB | | VSPTKLNDLCFT | 381 | 0.26 | 0.32 |
| | | D | | | | | |
| 48 | S | S1B/RB | S_006 | NDLCFTNVYADS | 387 | 0.47 | 0.54 |
| | | D | | | | | |
| 49 | S | S1B/RB | S_006 | NVYADSFVIRGD | 393 | 0.74 | 0.50 |
| | | D | | | | | |
| 50 | S | S1B/RB | | FVIRGDEVRQIA | 399 | 0.61 | 0.46 |
| | | D | | | | | |
| 51 | S | S1B/RB | | EVRQIAPGQTGK | 405 | 0.37 | 0.46 |
| | | D | | | | | |
| 52 | S | S1B/RB | | PGQTGKIADYNY | 411 | 0.64 | 0.55 |
| | | D | | | | | |
| 53 | S | S1B/RB | | IADYNYKLPDDF | 417 | 0.76 | 0.70 |
| | | D | | | | | |
| 54 | S | S1B/RB | S_007 | KLPDDFTGCVIA | 423 | 0.66 | 0.46 |
| | | D | | | | | |
| 55 | S | S1B/RB | | TGCVIAWNSNNL | 429 | 0.29 | 0.30 |
| | | D | | | | | |
| 56 | S | S1B/RB | | WNSNNLDSKVGG | 435 | 0.27 | 0.35 |
| | | D | | | | | |
| 57 | S | S1B/RB | | DSKVGGNYNYLY | 441 | 0.54 | 0.52 |
| | | D | | | | | |
| 58 | S | S1B/RB | | NYNYLYRLFRKS | 447 | 0.37 | 0.50 |
| | | D | | | | | |
| 59 | S | S1B/RB | | RLFRKSNLKPFE | 453 | 0.64 | 0.54 |
| | | D | | | | | |
| 60 | S | S1B/RB | S_008 | NLKPFERDISTE | 459 | 0.67 | 0.51 |
| | | D | | | | | |
| 61 | S | S1B/RB | | RDISTEIYQAGS | 465 | 0.44 | 0.60 |
| | | D | | | | | |
| 62 | S | S1B/RB | | IYQAGSTPCNGV | 471 | 0.40 | 0.32 |
| | | D | | | | | |
| 63 | S | S1B/RB | S_009 | TPCNGVEGENCY | 477 | 0.67 | 0.65 |
| | | D | | | | | |
| 64 | S | S1B/RB | S_009 | EGFNCYFPLQSY | 483 | 0.50 | 0.66 |
| | | D | | | | | |
| 65 | S | S1B/RB | | FPLQSYGFQPTN | 489 | 0.35 | 0.44 |
| | | D | | | | | |
| 66 | S | S1B/RB | | GFQPTNGVGYQP | 495 | 0.49 | 0.58 |
| | | D | | | | | |
| 67 | S | S1B/RB | | GVGYQPYRVVVL | 501 | 0.38 | 0.60 |
| | | D | | | | | |
| 68 | S | S1B/RB | | YRVVVLSFELLH | 507 | 0.42 | 0.46 |
| | | D | | | | | |
| 69 | S | S1B/RB | | SFELLHAPATVC | 513 | 0.44 | 0.37 |
| | | D | | | | | |
| 70 | S | S1B/RB | | APATVCGPKKST | 519 | 0.29 | 0.48 |
| | | D | | | | | |
| 71 | S | S1B/RB | | TVCGPKKSTNLV | 522 | 0.40 | 0.54 |
| | | D | | | | | |
| 72 | S | S1C | S_010 | LTGTGVLTESNK | 545 | 0.56 | 0.49 |
| 73 | S | S1C | S_010 | TGVLTESNKKFL | 548 | 0.48 | 0.58 |
| 74 | S | S1C | S_010 | LTESNKKFLPFQ | 551 | 0.61 | 0.53 |
| 75 | S | S1C | S_010 | SNKKFLPFQQFG | 554 | 0.44 | 0.28 |
| 76 | S | S1C | S_010 | KFLPFQQFGRDI | 557 | 0.51 | 0.58 |
| 7 | S | S1C | S_010 | PFQQFGRDIADT | 560 | 0.88 | 0.58 |
| 18 | S | S1C | S_010 | QFGRDIADTTDA | 563 | 0.80 | 0.73 |
| 77 | S | S1C | S_010 | RDIADTTDAVRD | 566 | 0.73 | 0.57 |
| 22 | S | S1C | S_010 | ADTTDAVRDPQT | 569 | 0.73 | 0.80 |
| 78 | S | S1C | S_010 | TTDAVRDPQTLE | 571 | 0.65 | 0.59 |
| 4 | S | S1C | S_010 | TDAVRDPQTLEI | 572 | 0.90 | 0.78 |
| 2 | S | S1C | S_010 | VRDPQTLEILDI | 575 | 0.94 | 0.80 |
| 79 | S | S1C | S_010 | DPQTLEILDITP | 577 | 0.57 | 0.46 |
| 80 | S | S1C | S_010 | PQTLEILDITPC | 578 | 0.63 | 0.56 |
| 81 | S | S1C | S_010 | LEILDITPCSFG | 581 | 0.52 | 0.53 |
| 82 | S | S1 | | ILDITPCSFGGV | 583 | 0.53 | 0.49 |
| 83 | S | S1 | | LDITPCSFGGVS | 584 | 0.38 | 0.48 |
| 84 | S | S1 | | TPCSFGGVSVIT | 587 | 0.47 | 0.50 |
| 85 | S | S1 | | SFGGVSVITPGT | 590 | 0.37 | 0.36 |
| 86 | S | S1D | S_011 | GVSVITPGTNTS | 593 | 0.35 | 0.55 |
| 87 | S | S1D | S_011 | VITPGTNTSNQV | 596 | 0.43 | 0.50 |
| 88 | S | S1D | S_011 | PGTNTSNQVAVL | 599 | 0.43 | 0.43 |
| 89 | S | S1D | S_011 | NTSNQVAVLYQD | 602 | 0.46 | 0.47 |
| 90 | S | S1D | S_011 | NQVAVLYQDVNC | 605 | 0.58 | 0.53 |
| 91 | S | S1D | S_011 | AVLYQDVNCTEV | 608 | 0.47 | 0.48 |
| 92 | S | S1D | S_011 | VLYQDVNCTEVP | 609 | 0.38 | 0.38 |
| 93 | S | S2 | S_015 | KRSFIEDLLFNK | 813 | 0.71 | 0.51 |
| 94 | S | S2 | S_015 | FIEDLLFNKVTL | 816 | 0.73 | 0.58 |
| 95 | S | S2 | S_015 | DLLFNKVTLADA | 819 | 0.48 | 0.50 |
| 96 | S | S2 | | FNKVTLADAGFI | 822 | 0.27 | 0.49 |
| 97 | S | S2 | | VTLADAGFIKQY | 825 | 0.54 | 0.60 |
| 98 | S | S2 | S_016 | ADAGFIKQYGDC | 828 | 0.65 | 0.47 |
| 99 | S | S2 | S_016 | DAGFIKQYGDCL | 829 | 0.45 | 0.39 |
| 100 | S | S2-HR1 | | ALGKLQDVVNQN | 943 | 0.50 | 0.49 |
| 101 | S | S2-HR1 | | KLQDVVNQNAQA | 946 | 0.36 | 0.42 |
| 102 | S | S2-HR1 | | DVVNQNAQALNT | 949 | 0.39 | 0.39 |
| 103 | S | S2-HR1 | | NQNAQALNTLVK | 952 | 0.34 | 0.43 |
| 104 | S | S2-HR1 | | AQALNTLVKQLS | 955 | 0.28 | 0.25 |
| 105 | S | S2-HR1 | | LNTLVKQLSSNF | 958 | 0.39 | 0.29 |
| 106 | S | S2-HR1 | | TLVKQLSSNFGA | 960 | 0.26 | 0.34 |
| 107 | S | S2 | S_019 | DPLQPELDSFKE | 1138 | 0.77 | 0.77 |
| 11 | S | S2 | S_019 | QPELDSFKEELD | 1141 | 0.84 | 0.77 |
| 108 | S | S2 | S_019 | LDSFKEELDKYF | 1144 | 0.65 | 0.63 |
| 109 | S | S2 | S_019 | FKEELDKYFKNH | 1147 | 0.50 | 0.52 |
| 110 | S | S2 | S_019 | ELDKYFKNHTSP | 1150 | 0.41 | 0.53 |
| 111 | S | S2 | S_019 | DKYFKNHTSPDV | 1152 | 0.53 | 0.37 |
| 112 | S | S2 | S_020 | GINASVVNIQKE | 1170 | 0.55 | 0.70 |
| 113 | S | S2 | S_020 | ASVVNIQKEIDR | 1173 | 0.73 | 0.52 |
| 114 | S | S2 | S_020 | VNIQKEIDRLNE | 1176 | 0.39 | 0.35 |
| 115 | S | S2 | S_020 | QKEIDRLNEVAK | 1179 | 0.64 | 0.64 |
| 116 | S | S2 | S_020 | IDRLNEVAKNLN | 1182 | 0.43 | 0.34 |
| 117 | S | S2 | S_020 | LNEVAKNLNESL | 1185 | 0.20 | 0.44 |
| 118 | S | S2 | S_020 | EVAKNLNESLID | 1187 | 0.51 | 0.63 |
| 119 | S | S2-CT | S_021 | CCSCGSCCKFDE | 1246 | 0.65 | 0.55 |
| 120 | S | S2-CT | S_021 | CGSCCKFDEDDS | 1249 | 0.77 | 0.70 |
| 6 | S | S2-CT | S_021 | CCKFDEDDSEPV | 1252 | 0.88 | 0.71 |
| 13 | S | S2-CT | S_021 | FDEDDSEPVLKG | 1255 | 0.83 | 0.87 |
| 121 | S | S2-CT | S_021 | DDSEPVLKGVKL | 1258 | 0.72 | 0.75 |
| 122 | S | S2-CT | S_021 | EPVLKGVKLHYT | 1261 | 0.35 | 0.53 |
| 123 | N | | N_001 | PRITFGGPSDST | 12 | 0.71 | 0.45 |
| 124 | N | | N_001 | TFGGPSDSTGSN | 15 | 0.67 | 0.50 |
| 125 | N | | N_001 | GPSDSTGSNQNG | 18 | 0.75 | 0.51 |
| 126 | N | | N_001 | DSTGSNQNGERS | 21 | 0.63 | 0.48 |
| 127 | N | | N_001 | GSNQNGERSGAR | 24 | 0.61 | 0.71 |
| 128 | N | | N_001 | QNGERSGARSKQ | 27 | 0.76 | 0.58 |
| 129 | N | | N_001 | ERSGARSKQRRP | 30 | 0.73 | 0.53 |
| 130 | N | | N_001 | GARSKQRRPQGL | 33 | 0.56 | 0.55 |
| 131 | N | | N_001 | RSKQRRPQGLPN | 35 | 0.63 | 0.40 |
| 132 | N | | N_005 | SQASSRSSSRSR | 179 | 0.44 | 0.44 |
| 133 | N | | N_005 | SSRSSSRSRNSS | 182 | 0.51 | 0.46 |
| 134 | N | | N_005 | SSSRSRNSSRNS | 185 | 0.46 | 0.46 |
| 135 | N | | N_005 | RSRNSSRNSTPG | 188 | 0.39 | 0.43 |
| 136 | N | | N_005 | NSSRNSTPGSSR | 191 | 0.27 | 0.53 |
| 137 | N | | | RNSTPGSSRGTS | 194 | 0.40 | 0.36 |
| 138 | N | | | NSTPGSSRGTSP | 195 | 0.33 | 0.36 |
| 139 | N | | N_006 | KMSGKGQQQQGQ | 232 | 0.59 | 0.46 |
| 19 | N | | N_006 | QQQQGQTVTKKS | 238 | 0.80 | 0.54 |
| 140 | N | | N_006 | TVTKKSAAEASK | 244 | 0.44 | 0.54 |
| 5 | N | | N_006 | AAEASKKPRQKR | 250 | 0.90 | 0.71 |
| 12 | N | | N_006 | AEASKKPRQKRT | 251 | 0.84 | 0.71 |
| 141 | N | | N_010 | YKTFPPTEPKKD | 359 | 0.77 | 0.71 |
| 3 | N | | N_010 | FPPTEPKKDKKK | 362 | 0.92 | 0.81 |
| 1 | N | | N_010 | TEPKKDKKKKAD | 365 | 0.94 | 0.81 |
| 142 | N | | N_010 | KKDKKKKADETQ | 368 | 0.73 | 0.65 |
| 143 | N | | N_010 | KKKKADETQALP | 371 | 0.59 | 0.48 |
| 144 | N | | N_010 | KADETQALPQRQ | 374 | 0.54 | 0.67 |
| 145 | N | | N_010 | ADETQALPQRQK | 375 | 0.61 | 0.59 |
| 146 | N | | N_010 | ETQALPQRQKKQ | 377 | 0.71 | 0.39 |
| 147 | N | | N_010 | QALPQRQKKQQT | 379 | 0.29 | 0.32 |
| 148 | M | | M_003 | GRCDIKDLPKEI | 156 | 0.63 | 0.58 |
| 149 | M | | M_003 | DLPKEITVATSR | 162 | 0.49 | 0.46 |
| 150 | M | | | TVATSRTLSYYK | 168 | 0.21 | 0.33 |
| 151 | M | | | TSRTLSYYKLGA | 171 | 0.33 | 0.27 |
| 17 | ORF1a | NSP1 | ORF1ab_ | LGDELGTDPYED | 144 | 0.81 | 0.75 |
| | b | | 005_ | | | | |
| 152 | ORF1a | NSP1 | ORF1ab_ | TDPYEDFQENWN | 150 | 0.69 | 0.62 |
| | b | | 005 | | | | |
| 153 | ORF1a | NSP1 | ORF1ab_ | FQENWNTKHSSG | 156 | 0.48 | 0.48 |
| | b | | 005 | | | | |
| 154 | ORF1a | NSP1 | ORF1ab | TKHSSGVTRELM | 162 | 0.57 | 0.58 |
| | b | | _005 | | | | |
| 155 | ORF1a | NSP1 | ORF1ab | KHSSGVTRELMR | 163 | 0.52 | 0.55 |
| | b | | _005 | | | | |
| 156 | ORF1a | NSP2 | ORF1ab | KRGVYCCREHEH | 224 | 0.69 | 0.52 |
| | b | | _007 | | | | |
| 157 | ORF1a | NSP2 | ORF1ab | CREHEHEIAWYT | 230 | 0.69 | 0.61 |
| | b | | _007 | | | | |
| 158 | ORF1a | NSP2 | ORF1ab | EIAWYTERSEKS | 236 | 0.58 | 0.58 |
| | b | | _007 | | | | |
| 159 | ORF1a | NSP2 | ORF1ab | ERSEKSYELQTP | 242 | 0.32 | 0.38 |
| | b | | _007 | | | | |
| 160 | ORF1a | NSP2 | ORF1ab | RSEKSYELQTPF | 243 | 0.55 | 0.47 |
| | b | | _007 | | | | |
| 16- | ORF1a | NSP3 | ORF1ab | ASHMYCSFYPPD | 916 | 0.73 | 0.53 |
| | b | | _018 | | | | |
| 162 | ORF1a | NSP3 | ORF1ab | YCSFYPPDEDEE | 920 | 0.76 | 0.74 |
| | b | | _018 | | | | |
| 163 | ORF1a | NSP3 | ORF1ab | SFYPPDEDEEEG | 922 | 0.74 | 0.65 |
| | b | | _018 | | | | |
| 20 | ORF1a | NSP3 | ORF1ab | PDEDEEEGDCEE | 926 | 0.79 | 0.83 |
| | b | | _018 | | | | |
| 164 | ORF1a | NSP3 | ORF1ab | EDEEEGDCEEEE | 928 | 0.78 | 0.78 |
| | b | | _018 | | | | |
| 16 | ORF1a | NSP3 | ORF1ab | EGDCEEEEFEPS | 932 | 0.81 | 0.80 |
| | b | | _018 | | | | |
| 165 | ORF1a | NSP3 | ORF1ab | DCEEEEFEPSTQ | 934 | 0.69 | 0.63 |
| | b | | _018 | | | | |
| 166 | ORF1a | NSP3 | ORF1ab | EEFEPSTQYEYG | 938 | 0.33 | 0.52 |
| | b | | _018 | | | | |
| 167 | ORF1a | NSP3 | ORF1ab | FEPSTQYEYGTE | 940 | 0.70 | 0.66 |
| | b | | _018 | | | | |
| 168 | ORF1a | NSP3 | ORF1ab | TQYEYGTEDDYQ | 944 | 0.75 | 0.64 |
| | b | | _018 | | | | |
| 169 | ORF1a | NSP3 | ORF1ab | YEYGTEDDYQGK | 946 | 0.58 | 0.64 |
| | b | | _018 | | | | |
| 170 | ORF1a | NSP3 | ORF1ab | EYGTEDDYQGKP | 947 | 0.61 | 0.60 |
| | b | | _018 | | | | |
| 14 | ORF1a | NSP3 | ORF1ab | TEDDYQGKPLEF | 950 | 0.83 | 0.65 |
| | b | | _018 | | | | |
| 171 | ORF1a | NSP3 | ORF1ab | GKPLEFGATSAA | 956 | 0.38 | 0.44 |
| | b | | _018 | | | | |
| 172 | ORF1a | NSP3 | ORF1ab | EFGATSAALQPE | 960 | 0.72 | 0.69 |
| | b | | _018 | | | | |
| 173 | ORF1a | NSP3 | ORF1ab | GATSAALQPEEE | 962 | 0.58 | 0.48 |
| | b | | _018 | | | | |
| 174 | ORF1a | NSP3 | ORF1ab | AALQPEEEQEED | 966 | 0.44 | 0.55 |
| | b | | _018 | | | | |
| 9 | ORF1a | NSP3 | ORF1ab | LQPEEEQEEDWL | 968 | 0.86 | 0.71 |
| | b | | _018 | | | | |
| 175 | ORF1a | NSP3 | ORF1ab | EEQEEDWLDDDS | 972 | 0.66 | 0.71 |
| | b | | _018 | | | | |
| 176 | ORF1a | NSP3 | ORF1ab | QEEDWLDDDSQQ | 974 | 0.61 | 0.64 |
| | b | | _018 | | | | |
| 177 | ORF1a | NSP3 | ORF1ab | WLDDDSQQTVGQ | 978 | 0.70 | 0.71 |
| | b | | _018 | | | | |
| 8 | ORF1a | NSP3 | ORF1ab | DDDSQQTVGQQD | 980 | 0.86 | 0.73 |
| | b | | _018 | | | | |
| 178 | ORF1a | NSP3 | ORF1ab | SQQTVGQQDGSE | 983 | 0.65 | 0.47 |
| | b | | _018 | | | | |
| 179 | ORF1a | NSP3 | | GPITDVFYKENS | 1860 | 0.57 | 0.49 |
| | b | | | | | | |
| 180 | ORF1a | NSP3 | | FYKENSYTTTIK | 1866 | 0.38 | 0.33 |
| | b | | | | | | |
| 181 | ORF1a | NSP3 | | YTTTIKPVTYKL | 1872 | 0.42 | 0.22 |
| | b | | | | | | |
| 182 | ORF1a | NSP3 | ORF1ab | PVTYKLDGVVCT | 1878 | 0.40 | 0.50 |
| | b | | _030 | | | | |
| 183 | ORF1a | NSP3 | ORF1ab | VTYKLDGVVCTE | 1879 | 0.54 | 0.54 |
| | b | | _030 | | | | |
| 10 | ORF1a | NSP11 | ORF1ab | DDDYFNKKDWYD | 4544 | 0.85 | 0.81 |
| | b | | _068 | | | | |
| 184 | ORF1a | NSP11 | ORF1ab | KKDWYDFVENPD | 4550 | 0.72 | 0.71 |
| | b | | _068 | | | | |
| 185 | ORF1a | NSP11 | ORF1ab | FVENPDILRVYA | 4556 | 0.74 | 0.64 |
| | b | | _068 | | | | |
| 186 | ORF1a | NSP11 | ORF1ab | ILRVYANLGERV | 4562 | 0.65 | 0.70 |
| | b | | _068 | | | | |
| 187 | ORF1a | NSP11 | | LRVYANLGERVR | 4563 | 0.52 | 0.39 |
| | b | | | | | | |
| 188 | ORF1a | NSP11 | ORF1ab | MLTNDNTSRYWE | 5297 | 0.68 | 0.65 |
| | b | | _084 | | | | |
| 189 | ORF1a | NSP11 | ORF1ab | TSRYWEPEFYEA | 5303 | 0.61 | 0.55 |
| | b | | _084 | | | | |
| 190 | ORF1a | NSP11 | ORF1ab | PEFYEAMYTPHT | 5309 | 0.63 | 0.66 |
| | b | | _084 | | | | |
| 191 | ORF1a | | | MYTPHTVLQAVG | 5315 | 0.44 | 0.48 |
| | b | | | | | | |
| 21 | ORF1a | NSP12 | ORF1ab | ETTADIVVFDEI | 5688 | 0.75 | 0.81 |
| | b | | _090 | | | | |
| 192 | ORF1a | NSP12 | ORF1ab | DIVVFDEISMAT | 5692 | 0.71 | 0.48 |
| | b | | _090 | | | | |
| 193 | ORF1a | NSP12 | ORF1ab | CRRCPAEIVDTV | 5764 | 0.69 | 0.42 |
| | b | | _092 | | | | |
| 194 | ORF1a | NSP12 | ORF1ab | EIVDTVSALVYD | 5770 | 0.50 | 0.61 |
| | b | | _092 | | | | |
| 195 | ORF1a | NSP12 | ORF1ab | SALVYDNKLKAH | 5776 | 0.40 | 0.66 |
| | b | | _092 | | | | |
| 196 | ORF1a | NSP12 | | NKLKAHKDKSAQ | 5782 | 0.51 | 0.52 |
| | b | | | | | | |
| 197 | ORF1a | NSP12 | ORF1ab | PTQTVDSSQGSE | 5852 | 0.71 | 0.66 |
| | b | | _094 | | | | |
| 198 | ORF1a | NSP12 | ORF1ab | SSQGSEYDYVIF | 5858 | 0.71 | 0.73 |
| | b | | _094 | | | | |
| 199 | ORF1a | NSP12 | ORF1ab | YDYVIFTQTTET | 5864 | 0.69 | 0.52 |
| | b | | _094 | | | | |
| 200 | ORF1a | NSP12 | | TQTTETAHSCNV | 5870 | 0.63 | 0.53 |
| | b | | | | | | |
| 201 | ORF1a | NSP12 | | TTETAHSCNVNR | 5872 | 0.44 | 0.42 |
| | b | | | | | | |
| 202 | ORF1a | NSP14 | ORF1ab | GLAKRFKESPFE | 6704 | 0.63 | 0.79 |
| | b | | _110 | | | | |
| 203 | ORF1a | NSP14 | ORF1ab | KESPFELEDFIP | 6710 | 0.77 | 0.72 |
| | b | | _110 | | | | |
| 204 | ORF1a | NSP14 | ORF1ab | LEDFIPMDSTVK | 6716 | 0.50 | 0.58 |
| | b | | _110 | | | | |
| 205 | ORF1a | NSP14 | | MDSTVKNYFITD | 6722 | 0.60 | 0.67 |
| | b | | | | | | |
| 206 | ORF1a | NSP14 | | DSTVKNYFITDA | 6723 | 0.55 | 0.48 |
| | b | | | | | | |
| 207 | ORF3a | | ORF3a | SGVVNPVMEPIY | 252 | 0.57 | 0.63 |
| | | | _003 | | | | |
| 208 | ORF3a | | ORF3a | VMEPIYDEPTTT | 258 | 0.67 | 0.64 |
| | | | _003 | | | | |
| 209 | ORF3a | | ORF3a | YDEPTTTTSVPL | 263 | 0.58 | 0.66 |
| | | | _003 | | | | |
| 210 | ORF8 | | | SLVVRCSFYEDF | 96 | 0.67 | 0.63 |
| 211 | ORF8 | | | SFYEDFLEYHDV | 102 | 0.70 | 0.77 |
| 212 | ORF8 | | | LEYHDVRVVLDF | 108 | 0.66 | 0.71 |
| 213 | ORF8 | | | EYHDVRVVLDFI | 109 | 0.52 | 0.58 |

¹AUC: Diagnostic accuracy (Receiver Operating Characteristic Area Under the Curve) for response of SARS-CoV-2-infected (n = 22) vs non-infected (n = 9) samples. Samples were tested individually against each peptide listed.

The most accurate diagnostic 3-peptide combinations for IgG-antibodies are:
SEQ ID NO 1 in combination with SEQ ID NO 2 and any one of SEQ ID NOS 7, 15, 18, 31, 35, 67, 113 and 139;
SEQ ID NOS 2, 74 and 128.

All these 3-peptide combinations reach an AUC of at least 0.99.

The most accurate diagnostic 3-peptide combinations for IgA-antibodies are any of the following combinations:
(vi) SEQ ID NO 2 in combination with SEQ ID NO 15 and any one of SEQ ID NOS 1 or 159;
(vii) SEQ ID NO 2 in combination with SEQ ID NOS 22 and 40;
(viii) SEQ ID NO 2 in combination with SEQ ID NOS 22 and 128;
(ix) SEQ ID NO 2 in combination with SEQ ID NOS 13 and 143;
(x) SEQ ID NO 2 in combination with SEQ ID NO 30 and 140.

All of the 3-peptide combinations mentioned hereinbefore reached an AUC of at least 0.90.

### Discussion

Musico et al recently reported twelve different 15-mer linear B-cell epitopes of SARS-COV-2 that may be useful for diagnosis (7). Although eight of those twelve peptides were among the epitopes the Applicant identified herein (epitopes S_001, S_008, S_009, N_004, ORF1ab_030, ORF1ab_056, ORF1ab_069 and ORF1ab_074), none was found among the most discriminatory peptides of the present invention (Table 4), indicating that the peptides of the present invention provide unique novel and inventive diagnostic opportunities.

Ladner et al recently reported a detailed profile of B-cell epitopes of SARS-COV-2 proteins S and N using a peptide library of 30-mer peptides (8). They identified three highly used epitopes in protein S (positions 560-572, 819-824 and 1150-1156), and three regions in protein N (positions 166-169, 223-229 and 390-402). Using the method of the invention, the Applicant has identified all these regions as epitopes in the current disclosure, and these regions are included in what the Applicant defines to be epitopes S_010, S_015, S_019, N_004, N_006 and N_010 (Table 1). Again, however, with the methodology of the invention technology, these particular epitope stretches are not among the most highly diagnostic epitopes (Table 4).

Shrock et al recently published a comprehensive mapping of SARS-COV-2 antibody responses using the VirScan technology, which uses a library of 50- and 20-mer peptides spanning the entire proteome of SARS-COV-2 (9). Shrock et al proposes a 3-peptide assay for accurate SARS-COV-2 diagnosis—two epitopes of protein S (positions 810-830 and 1146-1166) and one epitope in protein N (positions 386-406). These regions are defined by the Applicant as forming part of epitopes S_015, S_019 and N_010 herein. However, neither of those peptides are among the ones the Applicant have identified as being the most highly discriminatory in accordance with the present invention (Table 4). Shrock et al describe in total 823 distinct epitopes of SARS-COV-2, which is the most comprehensive mapping of linear B-cell epitopes to date. Among the 164 described epitopes of the present invention, 35% (n=57) were not described by Shrock et al., so the results presented herein advance SARS-COV-2 antibody diagnostics.

The Shrock et al and Ladner et al reports were generated using peptide libraries with longer peptides, as they were using 20-, 30- or 50-mer peptides analysed in suspension while the Applicant used 12-mer peptides immobilised onto a surface. The Applicant suggests that although the overlap in epitopes defined between the approach of the present invention and Shrock et al is encouraging, the discrepancies may be due to in which way the peptides are presented to the antibodies (suspension/phage display/array surface). The Applicant's inventive approach is a significant advantage since most immunoassays used for serology analysis utilise antigens/markers immobilised on a surface; the results from the present invention are therefore more reliable for use in development of antibody diagnostics and are more accurate.

The Applicant is also of the opinion that it is a superior approach to use shorter peptides for discovery of markers for diagnosis; since there is a considerable reactivity to SARS-CoV-2 peptides in pre-pandemic samples (Table 1 and see (9)), the use of longer peptides runs a higher risk of containing such cross-reactive stretches that would mask any diagnostics stretches in the peptides analysed. When the ultimate aim of the study is to develop a tool for clinical diagnostics, it is vitally important that the marker discovery phase of the work is carried out using a technology that presents the peptides in a way that is similar to the assay platform to be used for diagnosis.

Poh et al described two neutralising linear epitopes of protein S (4). The Applicant, using the methodology of the present invention, similarly identified these two epitopes in their comprehensive map as S_010 (contains S14P5 of Poh et al) and S_015/S_016 (contains most of S21P2 of Poh et al). The fact that linear epitopes may be neutralising paves the way for low-cost peptide-based precision diagnostics for neutralising antibodies.

In conclusion, the Applicant presents a comprehensive linear B-cell epitope map of the SARS-COV-2 proteome, consisting of 164 epitopes. Within this map the Applicant identified peptides that are highly useful for diagnosis of SARS-COV-2 infection if included as antigens in an antibody/serology test for SARS-COV-2, using the peptides and methodology of the present invention. These identified peptides can be used either alone or in combination of two, three, or more peptides of the invention, as described herein, to increase accuracy. Given that assay arrays can become expensive and uneconomical if larger peptides (or significant numbers of peptides) need to be included in such tests, the short peptides and high accuracy peptides of the present invention address significant shortcomings of the prior art in producing a suitably discriminatory method, combination of peptides, or diagnostic kit.

The Applicant is of the opinion that the present invention provides a new and useful diagnostic test, markers, and method for SARS-COV-2 infection diagnosis in subjects.

As such, the Applicant is of the opinion that they have identified a need for a diagnostic and differential test for SARS-COV-2 with improved diagnostic properties, for example improved specificity and sensitivity.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

It is to be appreciated that reference to “one example” or “an example” of the invention is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

It is to be understood that the terminology employed above is for the purpose of description and should not be regarded as limiting. The described embodiment is intended to be illustrative of the invention, without limiting the scope thereof. The invention is capable of being practised with various modifications and additions as will readily occur to those skilled in the art.

Various substantially and specifically practical and useful exemplary embodiments of the claimed subject matter are described herein, textually and/or graphically, including the best mode, if any, known to the inventors for carrying out the claimed subject matter. Variations (e.g. modifications and/or enhancements) of one or more embodiments described herein might become apparent to those of ordinary skill in the art upon reading this application.

The inventor(s) expects skilled artisans to employ such variations as appropriate, and the inventor(s) intends for the claimed subject matter to be practiced other than as specifically described herein. Accordingly, as permitted by law, the claimed subject matter includes and covers all equivalents of the claimed subject matter and all improvements to the claimed subject matter. Moreover, every combination of the above described elements, activities, and all possible variations thereof are encompassed by the claimed subject matter unless otherwise clearly indicated herein, clearly and specifically disclaimed, or otherwise clearly contradicted by context.

The use of any and all examples, or exemplary language (e.g., “such as”) provided herein, is intended merely to better illuminate one or more embodiments and does not pose a limitation on the scope of any claimed subject matter unless otherwise stated. No language in the specification should be construed as indicating any non-claimed subject matter as essential to the practice of the claimed subject matter.

The use of words that indicate orientation or direction of travel is not to be considered limiting. Thus, words such as “front”, “back”, “rear”, “side”, “up”, down”, “upper”, “lower”, “top”, “bottom”, “forwards”, “backwards”, “towards”, “distal”, “proximal”, “in”, “out” and synonyms, antonyms and derivatives thereof have been selected for convenience only, unless the context indicates otherwise. The inventor(s) envisage that various exemplary embodiments of the claimed subject matter can be supplied in any particular orientation and the claimed subject matter is intended to include such orientations.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “including,” “having,” “including,” and “containing” are to be construed as open-ended terms (i.e., meaning “including, but not limited to,”) unless otherwise noted.

Moreover, when any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate sub-range defined by such separate values is incorporated into the specification as if it were individually recited herein. For example, if a range of 1 to 10 is described, that range includes all values there between, such as for example, 1.1, 2.5, 3.335, 5, 6.179, 8.9999, and the like, and includes all sub-ranges there between, such as for example, 1 to 3.65, 2.8 to 8.14, 1.93 to 9, and the like.

Accordingly, every portion (e.g., title, field, background, summary, description, abstract, drawing figure, and the like.) of this application, other than the claims themselves, is to be regarded as illustrative in nature, and not as restrictive; and the scope of subject matter protected by any patent that issues based on this application is defined only by the claims of that patent.

### REFERENCES

1. Vita R, Mahajan S, Overton J A, Dhanda S K, Martini S, Cantrell J R, et al. The Immune Epitope Database (IEDB): 2018 update. Nucleic Acids Res. 2019 08; 47 (D1):D339-43.
2. Pedregosa F, Varoquaux G, Gramfort A, Michel V, Thirion B, Grisel O, et al. Scikit-learn: Machine Learning in Python. J Mach Learn Res. 2011; 12:2825-30.
3. Barnes C O, West A P, Huey-Tubman K E, Hoffmann M A G, Sharaf N G, Hoffman P R, et al. Structures of Human Antibodies Bound to SARS-COV-2 Spike Reveal Common Epitopes and Recurrent Features of Antibodies. Cell [Internet]. 2020 Jun. 24 [cited 2020 Aug. 5]; Available from: https://www.ncbi.nlm.nih.gov/pmc/articles/PMC7311918/4.
4. Poh C M, Carissimo G, Wang B, Amrun S N, Lee C Y-P, Chee R S-L, et al. Two linear epitopes on the SARS-COV-2 spike protein that elicit neutralising antibodies in COVID-19 patients. Nat Commun. 2020 Jun. 1; 11(1):2806.
5. Amrun S N, Lee C Y-P, Lee B, Fong S-W, Young B E, Chee R S-L, et al. Linear B-cell epitopes in the spike and nucleocapsid proteins as markers of SARS-COV-2 exposure and disease severity. EBioMedicine. 2020 August; 58:102911.
6. Yi Z, Ling Y, Zhang X, Chen J, Hu K, Wang Y, et al. Functional mapping of B-cell linear epitopes of SARS-COV-2 in COVID-19 convalescent population. Emerg Microbes Infect. 2020 December; 9(1):1988-96.
7. Musicò A, Frigerio R, Mussida A, Barzon L, Sinigaglia A, Riccetti S, et al. SARS-CoV-2 Epitope Mapping on Microarrays Highlights Strong Immune-Response to N Protein Region. Vaccines. 2021 January; 9(1):35.
8. Ladner J T, Henson S N, Boyle A S, Engelbrektson A L, Fink Z W, Rahee F, et al. Epitope-resolved profiling of the SARS-COV-2 antibody response identifies cross-reactivity with endemic human coronaviruses. Cell Rep Med [Internet]. 2021 Jan. 19 [cited 2021 Feb. 7]; 2(1). Available from: https://www.cell.com/cell-reports-5 medicine/abstract/S2666-3791(20)30244-5
9. Shrock E, Fujimura E, Kula T, Timms R T, Lee I-H, Leng Y, et al. Viral epitope profiling of COVID-19 patients reveals cross-reactivity and correlates of severity. Science [Internet]. 2020 Nov. 27 [cited 2021 Feb. 7]; 370(6520). Available from: https://science.sciencemag.org/content/370/6520/eabd4250

1. Use of at least one peptide sequence derived from a linear epitope of the SARS-COV-2 virus, for the identification of subjects infected with SARS-COV-2.

2. The use of claim 1, wherein the at least one peptide may be derived from a linear epitope of one or more of the S, N, or ORF1 proteins, or combinations thereof, for the identification of subjects infected with SARS-CoV-2.

3. Use of claim 1, wherein the peptide comprises or consists of an amino acid sequence selected from the group consisting of SEQ ID NO 1-22.

4. Use of claim 3, wherein the peptide comprises or consists of an amino acid sequence selected from the group consisting of SEQ ID NO 1-5.

5. A method of diagnosing a SARS-COV-2 infection in a subject or the detection of the presence of SARS-COV-2 in a subject, the method including the step of assaying a sample from the subject for the presence of antibodies that specifically bind to at least one peptide sequence derived from a linear epitope of any one or more of the S, N, or ORF1 proteins, or combinations thereof, of SARS-COV-2.

6. The method of claim 5, which includes the step of assaying for the presence of antibodies that specifically bind to one or more linear epitopes in the same SARS-COV-2 protein.

7. The method of claim 5, which includes the step of assaying for the presence of antibodies that specifically bind to one or more linear epitopes in different SARS-COV-2 proteins.

8. The method of claim 5, including the step of assaying a sample from the subject for the presence of antibodies that specifically bind to any one or more of the following epitopes, including combinations thereof, selected from the following:
in protein S, peptides within epitopes S_S005, S_S010, S_S019 and S_S021;
in protein N, peptides within epitopes N_006 and N_010;
in the ORF1ab polyprotein, peptides within epitopes ORF1a_005, ORF1a_018 and ORF1a_068.

9. The method of claim 8, wherein the method includes the step of assaying a sample from the subject for the presence of antibodies that specifically bind to any one or more of the peptides selected from the group comprising or consisting of:
SEQ ID NO 2, 4, 6, 7, 11, 13, 15 and 18;
SEQ ID NO 1, 3, 5, 12 and 19; and
SEQ ID NO 8, 9, 10, 14, 16 and 17.

10. The method of claim 5, which includes the step of assaying for the presence of antibodies that specifically bind to any one or more of the following epitopes, including combinations thereof, selected from the group comprising or consisting of:
S_005, S_010, S_021 (SEQ ID NOS 218, 223, 234);
N_010, ORF1ab_018, ORF1ab_068 (SEQ ID NOS 244, 267, 317); and
ORF1ab_090 (SEQ ID NO 229).

11. The method of claim 10, including the step of assaying for the presence of antibodies that specifically bind to any one or more of the peptides selected from the group consisting of:
SEQ ID NO 1, 2, 3, 10, 13, 15, 16, 20, 21 and 22.

12. A diagnostic assay or kit comprising a combination of 3 or more peptides, comprising peptides comprising or consisting of any one or more of the following combinations of amino acid sequences selected from the group consisting of:
SEQ ID NO 1 in combination with SEQ ID NO 2 and any one of SEQ ID NOS 7, 15, 18, 31, 35, 67, 113 and 139; and
SEQ ID NOS 2, 74 and 128.

13. A diagnostic assay or kit comprising a combination of 3 or more peptides, comprising peptides comprising or consisting of any one or more of the following combinations of amino acid sequences selected from the group consisting of:
SEQ ID NO 2 in combination with SEQ ID NO 15 and any one of SEQ ID NOS 1 or 159;
SEQ ID NO 2 in combination with SEQ ID NOS 22 and 40;
SEQ ID NO 2 in combination with SEQ ID NOS 22 and 128;
SEQ ID NO 2 in combination with SEQ ID NOS 13 and 143;
SEQ ID NO 2 in combination with SEQ ID NOS 30 and 140.

14. The use of claim 1, wherein the peptide sequence comprises at most 25 amino acids or fewer, preferably 20 amino acids or fewer, preferably 15 amino acids or fewer, most preferably 10 amino acids or fewer.

15. The method of claim 5, wherein the antibodies are IgG antibodies.

16. The method of claim 5, wherein the antibodies are IgA antibodies.

17. Use of claim 2, wherein the peptide comprises or consists of an amino acid sequence selected from the group consisting of SEQ ID NO 1-22.

18. The method of claim 6, including the step of assaying a sample from the subject for the presence of antibodies that specifically bind to any one or more of the following epitopes, including combinations thereof, selected from the following:
in protein S, peptides within epitopes S_S005, S_S010, S_S019 and S_S021;
in protein N, peptides within epitopes N_006 and N_010;
in the ORF1ab polyprotein, peptides within epitopes ORF1a_005, ORF1a_018 and ORF1a_068.

19. The method of claim 7, including the step of assaying a sample from the subject for the presence of antibodies that specifically bind to any one or more of the following epitopes, including combinations thereof, selected from the following:
in protein S, peptides within epitopes S_S005, S_S010, S_S019 and S_S021;
in protein N, peptides within epitopes N_006 and N_010;
in the ORF1ab polyprotein, peptides within epitopes ORF1a_005, ORF1a_018 and ORF1a_068.
